(19) United States
(12) Patent Application Publication
(10) Pub. No.: US 2014/0072523 A1
(43) Pub. Date: Mar. 13, 2014

(54) HAIR TREATMENT WITH SELECTED ESTEROLEN SILICONES AND ACRYLIC ACID DERIVATIVE WITH LOW WATER CONTENT

(71) Applicant: Henkel AG & Co. KGaA, Duesseldorf (DE)

(72) Inventors: Marlene Battermann, Asendorf (DE); Thomas Hippe, Appen (DE)

(73) Assignee: Henkel AG & Co. KGaA, Duesseldorf (DE)

(21) Appl. No.: 14/022,458

(22) Filed: Sep. 10, 2013

(30) Foreign Application Priority Data
Sep. 10, 2012 (DE) 10 2012 215 965.7

(51) Int. Cl. A61K 8/92; A61Q 5/12; A61K 8/891; A61K 8/37; A61K 8/892; A61Q 5/00; A61K 8/40

(52) U.S. Cl. CPC A61K 8/922; A61Q 5/00; A61Q 5/12; A61K 8/40; A61K 8/37; A61K 8/892; A61K 8/891
USPC 424/70.12

(57) ABSTRACT

Hair treatment agents include selected ester oils, selected silicones, and selected acrylic acid derivatives. The agents are used both for hair treatment and to improve shelf stability.

### FIELD OF THE INVENTION

The present invention generally relates to hair treatment agents containing selected ester oils, selected silicones, and selected acrylic acid derivatives, and to the use of said agents for hair treatment.

### BACKGROUND OF THE INVENTION

The significance of care-providing products having the longest-lasting possible effect is increasing, not least due to severe stress on the hair e.g. because of coloring or permanent waves, but also as a result of cleaning of the hair with shampoos and due to environmental stresses.

The known active substances, however, cannot meet all needs to a sufficient degree. A need therefore continues to exist for active substances or active substance combinations for cosmetic agents having good care-providing properties. Unstable compositions are often obtained especially when cosmetic oils such as silicones or glycerides are used, as well as natural oils such as sunflower oil. These instabilities turn out to be problematic in particular when the water content of the compositions is low. In such formulations, discolorations occur and the compositions prove not to be shelf-stable. The problem is that much greater when the compositions are intended to be clear, colorless, and transparent. Even with colored compositions, however, the color changes. This creates among consumers the impression that the product can no longer be used. In addition, the scent of the compositions is disadvantageously influenced.

It is therefore desirable to make available shelf-stable compositions, in particular stable in terms of color and scent, having a low water content.

Furthermore, other desirable features and characteristics of the present invention will become apparent from the subsequent detailed description of the invention and the appended claims, taken in conjunction with the accompanying drawings and this background of the invention.

### BRIEF SUMMARY OF THE INVENTION

It has now been found, surprisingly, that the above objectives are achieved by compositions containing selected ester oils, selected silicones, and selected acrylic acid derivatives, having a water content of at most 5.0 wt %.

At the same time, the use of these combinations results in surprisingly good properties for the treated hair, in particular improved combability and improved elasticity as well as appreciably enhanced washing fastness for colored hair, and to greater durability simultaneously with better reshaping performance in the context of waving operations such as water waving and permanent waving. In particular, however, the shine of the hair treated therewith is appreciably increased.

These positive properties are obtained with both a typical rinse-off and a leave-on application. The composition according to the present invention is also outstandingly suitable for spray application as a non-aerosol. An extraordinarily uniform spray pattern is achieved as compared with the hitherto usual products. As a result, the entire composition is distributed very uniformly on the hair.

The invention specifically includes a cosmetic composition for treating keratinic fibers, containing: at least one ester oil in a total quantity from 0.01 to 20.0 wt %; at least one silicone in a total quantity from 0.01 to 99 wt %; at least one acrylic acid derivative of formula c) in a total quantity from 0.01 to 5.0 wt %

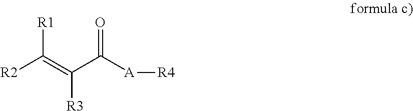

in which residues R1, R2, R3, mutually independently in each case, denote a residue selected from hydrogen, linear and/or branched, saturated and/or unsaturated C1 to C30 alkyl or alkenyl residues, in particular methyl, ethyl, propyl, isopropyl, butyl, isobutyl, tert-butyl, pentyl, amyl, isoamyl, neopentyl, hexyl, isohexyl, 2-ethylhexyl, heptanyl, isoheptanyl, octanyl, isooctanyl, nonyl, decyl, lauryl, myristyl, aryl, phenyl, methoxyphenyl, hydroxyphenyl, methoxybenzyl, hydroxybenzyl, benzyl, naphthyl, or —CN, A denotes O or —NR5, and R4 and optionally R5 mutually independently denote a residue selected from hydrogen, linear and/or branched, saturated and/or unsaturated C1 to C30 alkyl or alkenyl residues, in particular methyl, ethyl, propyl, isopropyl, butyl, isobutyl, tert-butyl, pentyl, amyl, isoamyl, neopentyl, hexyl, isohexyl, 2-ethylhexyl, heptanyl, isoheptanyl, octanyl, isooctanyl, nonyl, decyl, lauryl, myristyl, aryl, phenyl, methoxyphenyl, hydroxyphenyl, methoxybenzyl, hydroxybenzyl, benzyl, or naphthyl; and water in a total quantity from 0 to at most 5.0 wt %.

### DETAILED DESCRIPTION OF THE INVENTION

The following detailed description of the invention is merely exemplary in nature and is not intended to limit the invention or the application and uses of the invention. Furthermore, there is no intention to be bound by any theory presented in the preceding background of the invention or the following detailed description of the invention.

A first subject of the present invention is therefore a composition for treating keratinic fibers containing, based on the total composition of the agent,

a) at least one ester oil in a total quantity from 0.01 to 20.0 wt %,

b) at least one silicone in a total quantity from 0.01 to 99 wt %,

c) at least one acrylic acid derivative of formula c)

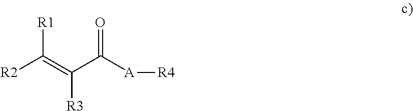

in which residues R1, R2, R3, mutually independently in each case, denote a residue selected from hydrogen, linear and/or branched, saturated and/or unsaturated C1 to C30 alkyl or alkenyl residues, in particular methyl, ethyl, propyl, isopropyl, butyl, isobutyl, tert-butyl, pentyl, amyl, isoamyl, neopentyl, hexyl, isohexyl, 2-ethylhexyl, heptanyl, isoheptanyl, octanyl, isooctanyl, nonyl, decyl, lauryl, myristyl, aryl, phenyl, methoxyphenyl, hydroxyphenyl, methoxybenzyl, hydroxybenzyl, benzyl, naphthyl, or —CN,
A denotes O or —NR5, and
R4 and optionally R5 mutually independently denote a residue selected from hydrogen, linear and/or branched, saturated and/or unsaturated C1 to C30 alkyl or alkenyl residues, in particular methyl, ethyl, propyl, isopropyl, butyl, isobutyl, tert-butyl, pentyl, amyl, isoamyl, neopentyl, hexyl, isohexyl, 2-ethylhexyl, heptanyl, isoheptanyl, octanyl, isooctanyl, nonyl, decyl, lauryl, myristyl, aryl, phenyl, methoxyphenyl, hydroxyphenyl, methoxybenzyl, hydroxybenzyl, benzyl, or naphthyl
in a total quantity from 0.01 to 5.0 wt %, and

d) water in a total quantity from 0 to at most 5.0 wt %.

The ingredients a) to d) are described below in detail. When “active substance complex (A)” is referred to below, this statement refers to the ingredients a) to d) obligatorily contained in the agents according to the present invention.

“Hair treatment agents” for purposes of the present invention are, for example, hair conditioners, hair sprays, hair rinses, hair treatments, hair packs, hair tonics, hair-setting agents, hair setting compositions, hair styling preparations, blow-dry lotions, foam setting agents, hair gels, hair waxes, or combinations thereof. Preferred agents according to the present invention are shampoos, conditioning agents, or hair tonics.

“Combability” is understood according to the present invention as both the combability of the wet fibers and the combability of the dry fibers. The combing work expended, or the force expended during the operation of combing an assemblage of fibers, serves as an indication of combability. The measurement parameters can be assessed sensorially by one skilled in the art, or quantified using measurement devices.

“Softness” is defined as the tactility of an assemblage of fibers, in which context the person skilled in the art sensorially feels and evaluates the “fullness” and “suppleness” parameters of the assemblage.

“Shapability” is understood as the ability to impart a change in shape to an assemblage of previously treated keratin-containing fibers, in particular human hairs. The term “stylability” is also used in hair cosmetics.

Suitable according to the present invention as cosmetic carriers are, in particular, OW, W/O, and W/O/W emulsions in the form of creams or gels or other preparations that are suitable in particular for use on the hair. The cosmetic carriers can be, in particular, aqueous or aqueous alcoholic.

“Aqueous alcoholic” cosmetic carriers are to be understood for purposes of the present invention as aqueous solutions containing 3 to 70 wt % of a C₁ to C₆ alcohol, in particular methanol, ethanol and/or propanol, isopropanol, butanol, isobutanol, tert-butanol, n-pentanol, isopentanols, n-hexanol, isohexanols, glycol, glycerol, 1,2-pentanediol, 1,5-pentanediol, 1,2-hexanediol, or 1,6-hexanediol. The agents according to the present invention can additionally contain further organic solvents such as, for example, methoxybutanol, benzyl alcohol, ethyl diglycol, or 1,2-propylene glycol. All water-soluble organic solvents are preferred in this context.

The total water content in the compositions according to the present invention is at most 5.0 wt %. A content of at most 3.0 wt % water is preferred. A content of at most 1.0 wt % water is particularly preferred. A water content of at most 0.3 wt % is highly preferred, and a water content of at most 0.1 wt % is most preferred.

For determination of the water content in the compositions according to the present invention, the water that may be contained in the raw materials used must also be taken into account. A consequence thereof is that all raw materials that contain a high water content should necessarily not be used in the compositions according to the present invention.

It is, however, possible according to the present invention to remove the water from such a raw material, if it is not obtainable with a reduced water content or in almost anhydrous fashion, using known methods, prior to use in the composition according to the present invention. Freeze-drying, distillation, etc. are suitable for this, for example.

The agents according to the present invention contain as ingredient a) at least one cosmetic oil selected from the ester oils. The ester oils preferably have a melting point lower than 50° C., particularly preferably lower than 45° C., very particularly lower than 40° C., highly preferably less than 35° C., and most preferably the cosmetic ester oils are flowable at a temperature less than 30° C. The ester oils are defined as follows:

“Ester oils” are to be understood as esters of C₆ to C₃₀ fatty acids with C₂ to C₃₀ fatty alcohols. The monoesters of fatty acids with alcohols having 2 to 24 carbon atoms are preferred. Examples of fatty-acid components used in the esters are hexanoic acid, octanoic acid, 2-ethylhexanoic acid, decanoic acid, lauric acid, isotridecanoic acid, myristic acid, palmitic acid, palmitoleic acid, stearic acid, isostearic acid, oleic acid, elaidic acid, petroselinic acid, linoleic acid, linolenic acid, eleostearic acid, arachidic acid, gadoleic acid, behenic acid, and erucic acid, as well as industrial mixtures thereof. Examples of the fatty-alcohol components in the ester oils are isopropyl alcohol, capronyl alcohol, capryl alcohol, 2-ethylhexyl alcohol, caprinyl alcohol, lauryl alcohol, isotridecyl alcohol, myristyl alcohol, cetyl alcohol, palmoleyl alcohol, stearyl alcohol, isostearyl alcohol, oleyl alcohol, elaidyl alcohol, petroselinyl alcohol, linolyl alcohol, linolenyl alcohol, eleostearyl alcohol, arachyl alcohol, gadoleyl alcohol, behenyl alcohol, erucyl alcohol, and brassidyl alcohol, as well as industrial mixtures thereof. Isopropyl myristate (Rilanit® IPM), isononanoic acid C16-18 alkyl esters (Cetiol® SN), 2-ethylhexyl palmitate (Cegesoft® 24), stearic acid 2-ethylhexyl ester (Cetiol® 868), cetyl oleate, glycerol tricaprylate, coconut fatty alcohol caprinate/caprylate (Cetiol® LC), n-butyl stearate, oleyl erucate (Cetiol® J 600), isopropyl palmitate (Rilanit® IPP), oleyl oleate (Cetiol®), lauric acid hexyl ester (Cetiol® A), di-n-butyl adipate (Cetiol® B), myristyl myristate (Cetiol® MM), cetearyl isononanoate (Cetiol® SN), oleic acid decyl ester (Cetiol® V) are particularly preferred according to the present invention.

The ester oils can of course also be alkoxylated with ethylene oxide, propylene oxide, or mixtures of ethylene oxide and propylene oxide. The alkoxylation can be located both on the fatty-alcohol part and on the fatty-acid part, and also on both parts, of the ester oils. It is preferred according to the present invention, however, if the fatty alcohol was first alkoxylated and then was esterified with fatty acid. Formula (D4-II) depicts these compounds in generalized fashion.

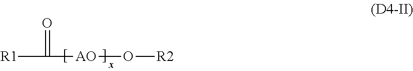

R1 here denotes a saturated or unsaturated, branched or unbranched, cyclic saturated or cyclic unsaturated acyl residue having 6 to 30 carbon atoms,
AO denotes ethylene oxide, propylene oxide, or butylene oxide,
X denotes a number between 1 and 200, by preference 1 and 100, particularly preferably between 1 and 50, very particularly preferably between 1 and 20, highly preferably between 1 and 10, and most preferably between 1 and 5,
R2 denotes a saturated or unsaturated, branched or unbranched, cyclic saturated or cyclic unsaturated alkyl, alkenyl, alkinyl, phenyl, or benzyl residue having 6 to 30 carbon atoms. Examples of fatty-acid components used as residue R1 in the esters are hexanoic acid, octanoic acid, 2-ethylhexanoic acid, decanoic acid, lauric acid, isotridecanoic acid, myristic acid, palmitic acid, palmitoleic acid, stearic acid, isostearic acid, oleic acid, elaidic acid, petroselinic acid, linoleic acid, linolenic acid, eleostearic acid, arachidic acid, gadoleic acid, behenic acid, and erucic acid, as well as industrial mixtures thereof. Examples of the fatty-alcohol components as residue R2 in the ester oils are benzyl alcohol, isopropyl alcohol, capronyl alcohol, capryl alcohol, 2-ethylhexyl alcohol, caprinyl alcohol, lauryl alcohol, isotridecyl alcohol, myristyl alcohol, cetyl alcohol, palmoleyl alcohol, stearyl alcohol, isostearyl alcohol, oleyl alcohol, elaidyl alcohol, petroselinyl alcohol, linolyl alcohol, linolenyl alcohol, eleostearyl alcohol, arachyl alcohol, gadoleyl alcohol, behenyl alcohol, erucyl alcohol, and brassidyl alcohol, as well as industrial mixtures thereof. An ester oil that is particularly preferred according to the present invention is obtainable, for example, under the INCI name PPG-3 Benzyl Ether Myristate.

Also to be understood as ester oils are:
dicarboxylic acid esters such as di-n-butyl adipate, di-(2-ethylhexyl) adipate, di-(2-ethylhexyl) succinate, and diisotridecyl acelaate, as well as diol esters such as ethylene glycol dioleate, ethylene glycol diisotridecanoate, propylene glycol di-(2-ethylhexanoate), propylene glycol diisostearate, propylene glycol dipelargonate, butanediol diisostearate, neopentyl glycol dicaprylate, as well as
symmetrical, asymmetrical, or cyclic esters of carbonic acid with fatty alcohols, for example glycerol carbonate or dicaprylyl carbonate (Cetiol® CC),
fatty acid triesters of saturated and/or unsaturated linear and/or branched fatty acids with glycerol,
fatty acid partial glycerides, i.e. monoglycerides, diglycerides, and industrial mixtures thereof. Typical examples are mono- and/or diglycerides based on hexanoic acid, octanoic acid, 2-ethylhexanoic acid, decanoic acid, lauric acid, isotridecanoic acid, myristic acid, palmitic acid, palmoleic acid, stearic acid, isostearic acid, oleic acid, elaidic acid, petroselinic acid, linoleic acid, linolenic acid, elaeostearic acid, arachidic acid, gadoleic acid, behenic acid and erucic acid, as well as industrial mixtures thereof. Oleic acid monoglycerides are preferably used.
Vegetable oils such as, for example, amaranth seed oil, apricot kernel oil, argan oil, avocado oil, babassu oil, cottonseed oil, borage seed oil, camelina oil, thistle oil, peanut oil, pomegranate seed oil, grapefruit seed oil, hemp oil, hazelnut oil, elderberry seed oil, blackcurrant seed oil, jojoba oil, cocoa butter, linseed oil, macadamia nut oil, corn oil, almond oil, marula oil, evening primrose oil, olive oil, orange oil, palm oil, peach kernel oil, rapeseed oil, rice oil, sea buckthorn pulp oil, sea buckthorn seed oil, sesame oil, shea butter, soy oil, sunflower oil, grapeseed oil, walnut oil, wheat germ oil, wild rose oil, and the liquid components of coconut oil.
Natural oils are, for example, other triglyceride oils that are not obtained on a vegetable basis, such as e.g. the liquid components of beef tallow, as well as synthetically manufactured triglyceride oils.

With particular preference, the ester oils are selected from the vegetable and/or natural oils. Highly preferably, the ester oils are selected from amaranth seed oil, apricot kernel oil, argan oil, avocado oil, babassu oil, cottonseed oil, borage seed oil, camelina oil, thistle oil, peanut oil, pomegranate seed oil, grapefruit seed oil, hemp oil, hazelnut oil, elderberry seed oil, blackcurrant seed oil, jojoba oil, cocoa butter, linseed oil, macadamia nut oil, corn oil, almond oil, marula oil, evening primrose oil, olive oil, orange oil, palm oil, peach kernel oil, rapeseed oil, rice oil, sea buckthorn pulp oil, sea buckthorn seed oil, sesame oil, shea butter, soy oil, sunflower oil, grapeseed oil, walnut oil, wheat germ oil, wild rose oil, and the liquid components of coconut oil, as well as mixtures of the aforementioned ester oils. Most preferably, the ester oils are selected from amaranth seed oil, apricot kernel oil, argan oil, avocado oil, babassu oil, cottonseed oil, pomegranate seed oil, grapefruit seed oil, hazelnut oil, elderberry seed oil, blackcurrant seed oil, jojoba oil, cocoa butter, macadamia nut oil, corn oil, almond oil, marula oil, evening primrose oil, olive oil, orange oil, sea buckthorn pulp oil, sea buckthorn seed oil, sesame oil, shea butter, soy oil, sunflower oil, grapeseed oil, walnut oil, wheat germ oil, wild rose oil, and mixtures thereof.

The ester oils are used in the agents according to the present invention in a quantity from 0.01 to 20 wt %, preferably 0.01 to 10.0 wt %, particularly preferably 0.01 to 7.5 wt %, highly preferably from 0.1 to 5.0 wt %. It is of course also possible according to the present invention to use several ester oils simultaneously.

Further oils according to the present invention that can optionally be used together with the ester oils and the silicones are:
Liquid paraffin oils, isoparaffin oils, and synthetic hydrocarbons, as well as di-n-alkyl ethers having a total of between 12 and 36 carbon atoms, in particular 12 to 24 carbon atoms, for example di-n-octyl ether, di-n-decyl ether, di-n-nonyl ether, di-n-undecyl ether, di-n-dodecyl ether, n-hexyl-n-octyl ether, n-octyl-n-decyl ether, n-decyl-n-undecyl ether, n-undecyl-n-dodecyl ether, and n-hexyl-n-undecyl ether, as well as di-tert-butyl ether, diisopentyl ether, di-3-ethyldecyl ether, tert-butyl-n-octyl ether, isopentyl-n-octyl ether, and 2-methylpentyl-n-octyl ether. The compounds 1,3-di-(2-ethylhexyl)cyclohexane (Cetiol® S) and di-n-octyl ether (Cetiol® OE), available as commercial products, may be preferred.

These oils just recited are used in the agents according to the present invention in a quantity from 0.01 to 20 wt %, preferably 0.01 to 10.0 wt %, particularly preferably 0.01 to 7.5 wt %, highly preferably from 0.1 to 5.0 wt %. It is of course also possible according to the present invention to use simultaneously several of these oils just recited.

In addition, ingredient b) of the compositions is at least one silicone oil. The silicone oil is selected in this context from the silicone oils known to one skilled in the art. The silicone oil is usually selected from the group of dimethicones, to which the cyclomethicones are also to be assigned, and/or from the group of dimethiconols and/or the group of the aminofunctional silicones. Although the dimethicone copolyols are also to be included among the silicone oils, it can be preferred according to the present invention not to select a dimethicone copolyol.

It is preferred according to the present invention if at least two different silicones are used. Most preferably, dimethicones, dimethiconols, cyclomethicones, and silicones of formula (Si-5), as well as mixtures thereof, are used. The silicones are used in a total quantity from at least 0.01 to 99.0 wt %. By preference, a total of 0.01 to 90.0 wt % silicones is used. Particularly preferably, the silicones are used in a total quantity from 0.1 to 85 wt %, based in each case on the total composition. Within this total quantity, the individual silicones to be used are utilized within the quantities recited in the context of the description of the individual silicones.

The dimethicones according to the present invention can be both linear and branched, and also cyclic or cyclic and branched. Linear dimethicones can be represented by the following structural formula (Si1):

(SiR¹₃)—O—(SiR²₂—O—)ₓ—(SiR¹₃) (Si1).

Branched dimethicones can be represented by the structural formula (Si1.1):

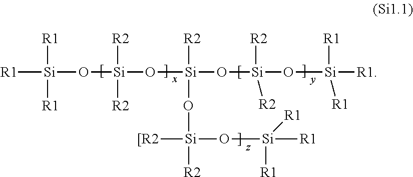

Residues R¹ and R² denote, mutually independently in each case, hydrogen, a methyl residue, a C2 to C30 linear, saturated or unsaturated hydrocarbon residue, a phenyl residue, and/or an aryl residue. The numbers x, y, and z are whole numbers and range, mutually independently in each case, from 0 to 50,000. The molecular weights of the dimethicones are between 1000 D and 10,000,000 D. The viscosities are between 100 and 10,000,000 cPs, measured at 25° C. using a glass capillary viscosimeter in accordance with Dow Corning Corporate Test Method CTM 0004 of Jul. 20, 1970. Preferred viscosities are between 1000 and 5,000,000 cPs; very particularly preferred viscosities are between 10,000 und 3,000,000 cPs. The most preferred range is between 50,000 und 2,000,000 cPs. Viscosities around the range of approximately 60,000 cPs are highly preferred. Reference may be made here, for example to the product “Dow Corning 200, 60,000 cSt.”

Particularly preferred cosmetic or dermatological preparations according to the present invention are characterized in that they contain at least one silicone of formula (Si1.2)

(CH₃)₃Si—[O—Si(CH₃)₂]ₓ—O—Si(CH₃)₃ (Si1.2),

in which x denotes a number from 0 to 100, by preference from 0 to 50, more preferably from 0 to 20, and in particular 0 to 10.

The dimethicones (Si1) are contained in the compositions according to the present invention in quantities from 0.01 to 10 wt %, by preference 0.01 to 8 wt %, particularly preferably 0.1 to 7.5 wt %, and in particular 0.1 to 5 wt %, based on the total composition.

Lastly, the dimethiconols (Si8) are understood as silicone compounds. The dimethiconols according to the present invention can be both linear and branched, and also cyclic or cyclic and branched. Linear dimethiconols can be represented by the following structural formula (Si8-I):

(SiOHR¹₂)—O—(SiR²₂—O—)ₓ—(SiOHR¹₂) (Si8-I).

Branched dimethiconols can be represented by the structural formula (Si8-II):

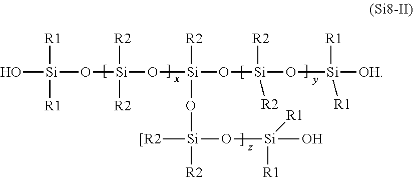

Residues R¹ and R² denote, mutually independently in each case, hydrogen, a methyl residue, a C2 to C30 linear, saturated or unsaturated hydrocarbon residue, a phenyl residue, and/or an aryl residue. The numbers x, y, and z are whole numbers and range, mutually independently in each case, from 0 to 50,000. The molecular weights of the dimethicones are between 1000 D and 10,000,000 D. The viscosities are between 100 and 10,000,000 cPs, measured at 25° C. using a glass capillary viscosimeter in accordance with Dow Corning Corporate Test Method CTM 0004 of Jul. 20, 1970. Preferred viscosities are between 1000 and 5,000,000 cPs; very particularly preferred viscosities are between 10,000 und 3,000,000 cPs. The most preferred range is between 50,000 und 2,000,000 cPs.

The following commercial products are recited as examples of such products: Dow Corning 1-1254 Fluid, Dow Corning 2-9023 Fluid, Dow Corning 2-9026 Fluid, Abil OSW 5 (Degussa Care Specialties), Dow Corning 1401 Fluid, Dow Corning 1403 Fluid, Dow Corning 1501 Fluid, Dow Corning 1784 HVF Emulsion, Dow Corning 9546 Silicone Elastomer Blend, SM555, SM2725, SM2765, SM2785 (all four aforesaid GE Silicones), Wacker-Belsil CM 1000, Wacker-Belsil CM 3092, Wacker-Belsil CM 5040, Wacker-Belsil DM 3096, Wacker-Belsil DM 3112 VP, Wacker-Belsil DM 8005 VP, Wacker-Belsil DM 60081 VP (all the aforesaid Wacker-Chemie GmbH).

The dimethiconols (Si8) are in the compositions according to the present invention in quantities from 0.01 to 95 wt %, by preference 0.01 to 70.0 wt %, particularly preferably 0.1 to 50.0 wt %, and in particular 0.1 to 35.0 wt % dimethiconol, based on the composition.

The cyclic dimethicones referred to according to INCI as Cyclomethicones are also usable with preference according to the present invention. Preferred here are cosmetic or dermatological preparations according to the present invention that contain at least one silicone of formula (Si-4)

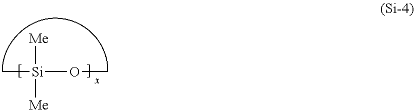

in which x denotes a number from 3 to 200, by preference from 3 to 10, more preferably from 3 to 7, and in particular 3, 4, 5, or 6.

The cyclomethicones (Si-4) are in the compositions according to the present invention in quantities from 0.1 to 95 wt %, by preference 0.1 to 85.0 wt %, particularly preferably 0.1 to 75.0 wt %, and in particular 1.0 to 70.0 wt % cyclomethicone, based on the composition.

Agents likewise preferred according to the present invention are characterized in that they contain at least one silicone of formula (Si-5)

R₃Si—[O—SiR₂]ₓ—(CH₂)ₙ—[O—SiR₂]_{y}—O—SiR₃ (Si-5),

in which R denotes identical or different residues from the group —H, phenyl, benzyl, —CH₂—CH(CH₃)Ph, C₁₋₂₀ alkyl residues, by preference —CH₃, —CH₂CH₃, —CH₂CH₂CH₃, —CH(CH₃)₂, —CH₂CH₂CH₂H₃, —CH₂CH(CH₃)₂, —CH(CH₃)CH₂CH₃, —C(CH₃)₃, x and/or y denotes a number from 0 to 200, by preference from 0 to 10, more preferably from 0 to 7, and in particular 0, 1, 2, 3, 4, 5, or 6, and n denotes a number from 0 to 10, preferably from 1 to 8, and in particular 2, 3, 4, 5, 6. The compound having the name Octamethyltrisiloxane is highly preferred in particular.

The silicones of formula (Si-5) are contained in the compositions according to the present invention in quantities from 0.1 to 75 wt %, by preference 0.1 to 60.0 wt %, particularly preferably 0.1 to 50.0 wt %, and in particular 1.0 to 40.0 wt %, based on the composition.

In particularly preferred embodiment of the active substance complex according to the present invention, the agents according to the present invention preferably contain at least one aminofunctional silicone.

Such silicones can be described, for example, by formula (Si-2)

M(RₐQ_{b}SiO_{(4-a-b)/2})ₓ(R_{c}SiO_{(4-c)/2})_{y}M (Si-2);

in the above formula,
R is a hydrocarbon or a hydrocarbon residue having 1 to approximately 6 carbon atoms,
Q is a polar residue of the general formula □R¹HZ, in which
   R¹ is a divalent connecting group that is bound to hydrogen and to the Z residue, assembled from carbon and hydrogen atoms, carbon, hydrogen, and oxygen atoms, or carbon, hydrogen, and nitrogen atoms, and
   Z is an organic aminofunctional residue that contains at least one aminofunctional group;
a assumes values in the range from approximately 0 to approximately 2,
b assumes values in the range from approximately 1 to approximately 3,
a+b is less than or equal to 3, and
c is a number in the range from approximately 1 to approximately 3, and
x is a number in the range from 1 to approximately 2,000, preferably from approximately 3 to approximately 50, and most preferably from approximately 3 to approximately 25, and
y is a number in the range from approximately 20 to approximately 10,000, by preference from approximately 125 to approximately 10,000, and most preferably from approximately 150 to approximately 1,000, and
M is a suitable silicone terminal group that is known in the existing art, by preference trimethylsiloxy.

Z according to formula (Si-2) is an organic aminofunctional residue containing at least one functional amino group. One possible formula for the aforesaid Z is NH(CH₂)_{z}NH₂, in which z is a whole number greater than or equal to 1. Another possible formula for the aforesaid Z is —NH(CH₂)_{z}(CH₂)_{zz}NH, in which both z and zz, mutually independently, are a whole number greater than or equal to 1, said structure encompassing diamino ring structures such as piperazinyl. The aforesaid Z is most preferably an —NHCH₂CH₂NH₂ residue. Another possible formula for the aforesaid Z is —N(CH₂)_{z}(CH₂)_{zz}NX₂ or —NX₂, in which each X of X₂ is selected independently from the group consisting of hydrogen and alkyl groups having 1 to 12 carbon atoms, and zz is 0.

Q according to formula (Si-2) is most preferably a polar aminofunctional residue of the formula —CH₂CH₂CH₂NHCH₂CH₂NH₂.

In formula (Si-2), a assumes values in the range from 0 to 2, b assumes values in the range from 2 to 3, a+b is less than or equal to 3, and c is a number in the range from 1 to 3.

Cationic silicone oils such as, for example, the commercially obtainable products Dow Corning (DC) 929 Emulsion, DC2-2078, DC5-7113, SM-2059 (General Electric), and SLM-55067 (Wacker) are suitable according to the present invention.

Particularly preferred agents according to the present invention are characterized in that they contain at least one aminofunctional silicone of formula (Si3-a)

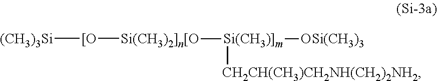

in which m and n are numbers whose sum (m+n) is between 1 and 2000, by preference between 50 and 150, where n by preference assumes values from 0 to 1999 and in particular from 49 to 149, and m by preference assumes values from 1 to 2000, in particular from 1 to 10.

These silicones are referred to according to the INCI declaration as Trimethylsilylaminodimethicones and are obtainable, for example, under the designation Q2-7224 (manufacturer: Dow Corning; a stabilized trimethylsilylamodimethicone).

Also particularly preferred are agents according to the present invention that contain at least one aminofunctional silicone of formula (Si-3b)

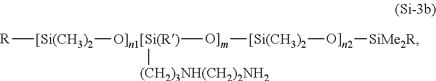

in which
R denotes —OH, an (optionally ethoxylated and/or propoxylated) (C₁ to C₂₀) alkoxy group, or a —CH₃ group,
R′ denotes —OH, a (C₁ to C₂₀) alkoxy group, or a —CH₃ group, and
m, n1, and n2 are numbers whose sum (m+n1+n2) is between 1 and 2000, by preference between 50 and 150, where the sum (n1+n2) by preference assumes values from 0 to 1999 and in particular from 49 to 149, and m by preference assumes values from 1 to 2000, in particular from 1 to 10.

These silicones are referred to according to the INCI declaration as Amodimethicones or as functionalized Amodimethicones, for example Bis (C13-15 Alkoxy) PG Amodimethicone (obtainable e.g. as a commercial product: DC 8500 of the Dow Corning company).

Suitable diquaternary silicones are selected from compounds of the general formula (Si3c)

[R¹R²R³N⁺-A-SiR⁷R⁸—(O—SiR⁹R¹⁰)ₙ—O—SiR¹¹R¹²-A-N⁺R⁴R⁵R⁶]2X⁻ (Si3c)

where residues R1 to R6 mutually independently signify C1 to C22 alkyl residues that can contain hydroxy groups, and where by preference at least one of the residues comprises at least 8 carbon atoms and the remaining residues comprise 1 to 4 carbon atoms,
residues R7 to R12 mutually independently are identical or different another and signify C1 to C10 alkyl or phenyl, A signifies a divalent organic connecting group, n is a number from 0 to 200, by preference from 10 to 120, particularly preferably from 10 to 40, and X⁻ is an anion.

The divalent connecting group is by preference a C1 to C12 alkylene or alkoxyalkylene group that can be substituted with one or more hydroxyl groups.

Particularly preferably, the group is —(CH₂)₃—O—CH₂—CH(OH)—CH₂—.

The anion X⁻ can be a halide ion, an acetate, an organic carboxylate, or a compound of the general formula RSO₃⁻, in which R has the meaning of C1 to C4 alkyl residues.

A very particularly preferred aminofunctional aminosilicone is at least one 4-morpholinomethyl-substituted silicone of formula (V)

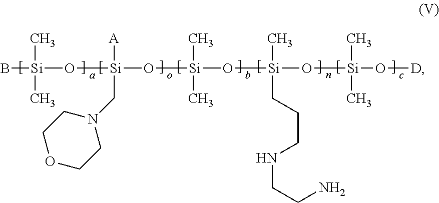

in which
A denotes a structural unit (I), (II), or (III) bound via —O—

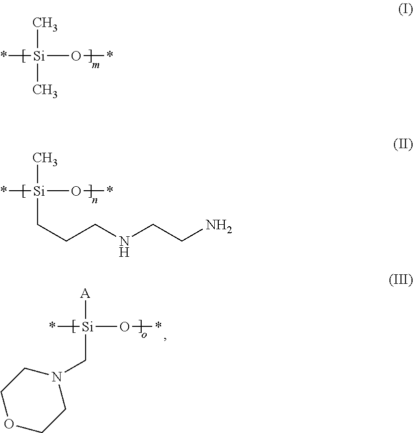

or an oligomeric or polymeric residue, bound via —O—, containing structural units of formulas (I), (II), or (III), or half of a connecting oxygen atom to a structural unit (III), or denotes —OH,
* denotes a bond to one of the structural units (I), (II), or (III), or denotes a terminal group B (Si-bound) or D (O-bound),
B denotes an —OH, —O—Si(CH₃)₃, —O—Si(CH₃)₂OH, —O—Si(CH₃)₂OCH₃ group,
D denotes an —H, —Si(CH₃)₃, —Si(CH₃)₂OH, —Si(CH₃)₂OCH₃ group,
a, b, and c denote whole numbers between 0 and 1000, with the provision that a+b+c>0,
m, n, and o denote whole numbers between 1 and 1000.

A preferred diquaternary silicone has the general formula (Si3d)

[RN⁺Me₂-A-(SiMe₂O)ₙ—SiMe₂-A-N⁺Me₂R]2CH₃COO⁻ (Si3d),

where A is the group —(CH₂)₃—O—CH₂—CH(OH)—CH₂, R is an alkyl residue having at least 8 carbon atoms, and n is a number from 10 to 120.

Suitable silicone polymers having two terminal quaternary ammonium groups are known under the INCI name Quatemium-80. These are dimethylsiloxanes having two terminal trialkylammonium groups. Diquaternary polydimethylsiloxanes of this kind are marketed by the Evonik company under the commercial names Abil® Quat 3270, 3272, and 3474.

Hair treatment agents preferred according to the present invention are characterized in that they contain, based on their weight, 0.01 to 10 wt %, by preference 0.01 to 8 wt %, particularly preferably 0.1 to 7.5 wt %, and in particular 0.2 to 5 wt % aminofunctional silicone(s) and/or diquaternary silicone.

Polyammonium-polysiloxane compounds are a further silicone according to the present invention having amino functions. Polyammonium-polysiloxane compounds can be acquired, for example, from GE Bayer Silicones under the commercial name Baysilone®. The products having the designations Baysilone TP 3911, SME 253, and SFE 839 are preferred in this context. It is very particularly preferred to use Baysilone TP 3911 as an active component of the compositions according to the present invention. Polyammonium-polysiloxane compounds are used in the compositions according to the present invention in a quantity from 0.01 to 10 wt %, by preference 0.01 to 7.5, particularly preferably 0.01 to 5.0 wt %, very particularly preferably from 0.05 to 2.5 wt %, referring in each case to the total composition.

EP 1887024 A1 describes novel cationic aminofunctional silicones that in particular improve shine in agents for the care of surfaces, for example human hair. These cationic silicone polymers are notable for the fact that they comprise a silicone skeleton as well as at least one polyether part and furthermore at least one part having an ammonium structure. Examples of the preferred cationic silicone polymers for purposes of the present invention are, besides the compounds of the aforementioned EP 1887024 A1 furthermore in particular the compounds having the INCI names: Silicone Quaternium-1, Silicone Quaternium-2, Silicone Quaternium-3, Silicone Quaternium-4, Silicone Quaternium-5, Silicone Quaternium-6, Silicone Quaternium-7, Silicone Quaternium-8, Silicone Quatemium-9, Silicone Quaternium-10, Silicone Quatemium-11, Silicone Quatemium-12, Silicone Quatemium-15, Silicone Quatemium-16, Silicone Quatemium-17, Silicone Quatemium-18, Silicone Quaternium-20, Silicone Quaternium-21, Silicone Quatemium-22, as well as Silicone Quaternium-2 Panthenol Succinate and Silicone Quatemium-16/Glycidyl Dimethicone Crosspolymer. Silicone Quatemium-22 is, in particular, most preferred. This raw material is marketed, for example, by the Evonik company under the commercial name Abil® T-Quat 60.

A further aminosilicone particularly preferred according to the present invention corresponds to the following formula:

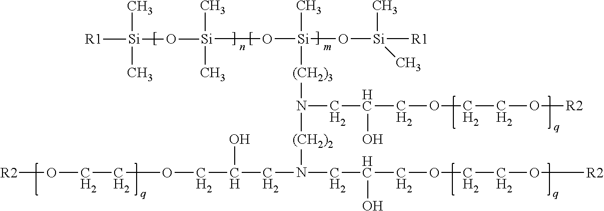

in which R1 denotes a methyl, ethyl, hydroxy, methoxy, or ethoxy group,
R2 denotes a straight-chain or branched C8 to C24 alkyl or alkenyl residue, by preference a straight-chain or branched C9 to C22 alkyl or alkenyl residue, particularly preferably a straight-chain or branched C11 to C18 alkyl or alkenyl residue, highly preferably a corresponding alkyl residue,
n and m each denote whole numbers from 1 to 1000, and
q respectively denotes a whole number from 2 to 50, preferably 4 to 30, particularly preferably 4 to 18, and highly preferably from 4 to 12.

The molecular weight of such compounds is 15,000 to 2,000,000, measured with a Brookfield RV rotary viscosimeter, spindle 5, at 20° C. The molar weight is preferably 30,000 to 1,750,000 and particularly preferably 50,000 to 1,500,000. The nitrogen content of the silicones according to the present invention is 0.03 to 4.2 wt %, preferably 0.1 to 2.8 wt %, and highly preferably 0.16 to 1.4 wt %. Aminofunctional cationic silicones according to the present invention of the above formula can be acquired, for example, from the Clariant company. A product highly preferred according to the present invention is commercially obtainable under the INCI name “Trideceth-9-Amodimethicone and Trideceth-12.”

The cationic aminofunctional silicone polymers of the formula described above are contained in the compositions according to the present invention in quantities from 0.01 to 5 wt %, preferably in quantities from 0.05 to 5 wt %, and very particularly preferably in quantities from 0.1 to 5 wt %. The best results of all are obtained with quantities from 0.1 to 2.5 wt %, based in each case on the total composition of the respective agent.

In general, the cationic aminofunctional silicones are contained in the compositions according to the present invention in quantities from 0.01 to 10 wt %, preferably in quantities from 0.05 to 10 wt %, and very particularly preferably in quantities from 0.1 to 7.5 wt %. The best results of all are obtained with quantities from 0.1 to 5 wt %, based in each case on the total composition of the respective agent.

Besides the dimethicones, dimethiconols, cyclomethicones, and/or amodimethicones according to the present invention, water-soluble silicones can be contained in the compositions according to the present invention as further silicones.

Corresponding hydrophilic silicones are selected, for example, from compounds of the formulas (Si-6) and/or (Si-7). In particular, preferred silicone-based water-soluble surfactants are selected from the group of dimethicone copolyols, which are preferably alkoxylated, in particular polyethoxylated or polypropoxylated.

“Dimethicone copolyols” are understood according to the present invention preferably as polyoxyalkylene-modified dimethylpolysiloxanes of the general formulas (Si-6) or (Si-7):

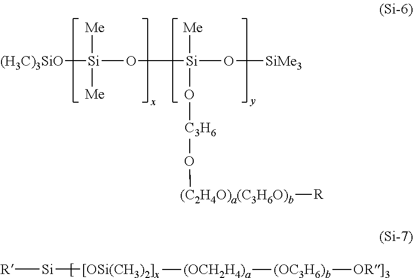

in which residue R denotes a hydrogen atom, an alkyl group having 1 to 12 carbon atoms, an alkoxy group having 1 to 12 carbon atoms, or a hydroxyl group, residues R′ and R″ signify alkyl groups having 1 to 12 carbon atoms, x denotes a whole number from 1 to 100, preferably from 20 to 30, y denotes a whole number from 1 to 20, preferably from 2 to 10, and a and b denote whole numbers from 0 to 50, preferably from 10 to 30.

Particularly preferred dimethicone polyols for purposes of the invention are, for example, the products marketed commercially under the trade name SILWET (Union Carbide Corporation) and DOW CORNING. Dimethicone copolyols particularly preferred according to the present invention are Dow Corning 190 and Dow Corning 193.

The dimethicone copolyols are in the compositions according to the present invention in quantities from 0.01 to 10 wt %, by preference 0.01 to 8 wt %, particularly preferably 0.1 to 7.5 wt %, and in particular 0.1 to 5 wt % dimethicone copolyol based on the composition.

The ratio of the quantities of all silicones to the quantity of all ester oils is from 1000:1 to 50:1, by preference from 500:1 to 50:1, and particularly preferably from 200:1 to 50:1 and highly preferably from 150:1 to 50:1.

The ingredient c) according to the present invention is a derivative of acrylic acid. Polymers that contain acrylic acid and acrylic acid derivatives are not included in the definition of the derivatives of acrylic acid. “Derivatives of acrylic acid” are to be understood in particular as the compounds described in formula c) below

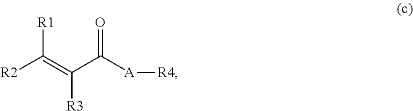

in which residues R1, R2, R3, mutually independently in each case, denote a residue selected from hydrogen, linear and/or branched, saturated and/or unsaturated C1 to C30 alkyl or alkenyl residues, in particular methyl, ethyl, propyl, isopropyl, butyl, isobutyl, tert-butyl, pentyl, amyl, isoamyl, neopentyl, hexyl, isohexyl, 2-ethylhexyl, heptanyl, isoheptanyl, octanyl, isooctanyl, nonyl, decyl, lauryl, myristyl, aryl, phenyl, methoxyphenyl, hydroxyphenyl, methoxybenzyl, hydroxybenzyl, benzyl, naphthyl, or —CN,
A denotes O or —NR5, and
R4 and optionally R5 mutually independently denote a residue selected from hydrogen, linear and/or branched, saturated and/or unsaturated C1 to C30 alkyl or alkenyl residues, in particular methyl, ethyl, propyl, isopropyl, butyl, isobutyl, tert-butyl, pentyl, amyl, isoamyl, neopentyl, hexyl, isohexyl, 2-ethylhexyl, heptanyl, isoheptanyl, octanyl, isooctanyl, nonyl, decyl, lauryl, myristyl, aryl, phenyl, methoxyphenyl, hydroxyphenyl, methoxybenzyl, hydroxybenzyl, benzyl, or naphthyl.

“A” preferably denotes oxygen, and the compounds conform to formula (c1)

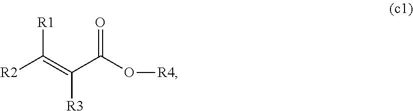

in which residues R1, R2, R3, mutually independently in each case, can denote a residue selected from hydrogen, linear and/or branched, saturated and/or unsaturated C1 to C30 alkyl or alkenyl residues, in particular methyl, ethyl, propyl, isopropyl, butyl, isobutyl, test-butyl, pentyl, amyl, isoamyl, neopentyl, hexyl, isohexyl, 2-ethylhexyl, heptanyl, isoheptanyl, octanyl, isooctanyl, nonyl, decyl, lauryl, myristyl, aryl, phenyl, methoxyphenyl, hydroxyphenyl, methoxybenzyl, hydroxybenzyl, benzyl, naphthyl, or —CN. R4 is selected from hydrogen, linear and/or branched, saturated and/or unsaturated C1 to C30 alkyl or alkenyl residues, in particular methyl, ethyl, propyl, isopropyl, butyl, isobutyl, tert-butyl, pentyl, amyl, isoamyl, neopentyl, hexyl, isohexyl, 2-ethylhexyl, heptanyl, isoheptanyl, octanyl, isooctanyl, nonyl, decyl, lauryl, myristyl, aryl, phenyl, methoxyphenyl, hydroxyphenyl, methoxybenzyl, hydroxybenzyl, benzyl, or naphthyl.

Preferred compounds of formula (c1) are notable for the fact that at least one of residues R1, R2, or R3 denotes a residue selected from hydrogen, aryl, phenyl, methoxyphenyl, hydroxyphenyl, methoxybenzyl, hydroxybenzyl, benzyl, or naphthyl, and residue R4 is selected from methyl, ethyl, propyl, isopropyl, butyl, isobutyl, tert-butyl, pentyl, amyl, isoamyl, neopentyl, hexyl, isohexyl, 2-ethylhexyl, heptanyl, isoheptanyl, octanyl, isooctanyl, nonyl, decyl, lauryl, or myristyl. Compounds of formula (c1) are highly preferred when at least two of residues R1, R2, or R3 denote hydrogen and the remaining residue denotes a residue selected from aryl, phenyl, methoxyphenyl, hydroxyphenyl, methoxybenzyl, hydroxybenzyl, benzyl, or naphthyl, preferably phenyl or methoxyphenyl, and residue R4 denotes a residue selected from methyl, ethyl, propyl, isopropyl, butyl, isobutyl, tert-butyl, pentyl, amyl, isoamyl, neopentyl, hexyl, isohexyl, 2-ethylhexyl, heptanyl, isoheptanyl, octanyl, isooctanyl, nonyl, decyl, lauryl, or myristyl, preferably isoamyl or ethylhexyl.

Further highly preferred compounds of formula (c1) are notable for the fact that at least two of residues R1, R2, or R3 denote a residue selected from hydrogen, aryl, phenyl, methoxyphenyl, hydroxyphenyl, methoxybenzyl, hydroxybenzyl, benzyl, or naphthyl, and residue R4 is selected from methyl, ethyl, propyl, isopropyl, butyl, isobutyl, tert-butyl, pentyl, amyl, isoamyl, neopentyl, hexyl, isohexyl, 2-ethylhexyl, heptanyl, isoheptanyl, octanyl, isooctanyl, nonyl, decyl, lauryl, or myristyl.

Very particularly preferred compounds of formula (c1) are characterized as follows:

R1 and R2 denote, mutually independently, a residue selected from hydrogen, aryl, phenyl, methoxyphenyl, hydroxyphenyl, methoxybenzyl, hydroxybenzyl, benzyl, or naphthyl, preferably phenyl or methoxyphenyl,
R3 denotes hydrogen or —CN, and R4 denotes a residue selected from methyl, ethyl, propyl, isopropyl, butyl, isobutyl, tert-butyl, pentyl, amyl, isoamyl, neopentyl, hexyl, isohexyl, 2-ethylhexyl, heptanyl, isoheptanyl, octanyl, isooctanyl, nonyl, decyl, lauryl, or myristyl, preferably isoamyl or ethylhexyl.

Highly preferred compounds of formula (c1) are characterized in that R1=R2 and are selected from hydrogen, aryl, phenyl, methoxyphenyl, hydroxyphenyl, methoxybenzyl, hydroxybenzyl, benzyl, or naphthyl, preferably phenyl or methoxyphenyl,

R3 denotes hydrogen or —CN, and R4 denotes a residue selected from methyl, ethyl, propyl, isopropyl, butyl, isobutyl, tert-butyl, pentyl, amyl, isoamyl, neopentyl, hexyl, isohexyl, 2-ethylhexyl, heptanyl, isoheptanyl, octanyl, isooctanyl, nonyl, decyl, lauryl, or myristyl, preferably isoamyl or ethylhexyl.

The most preferred compound of formula (c1) is that in which R1=R2=phenyl, R3=—CN, and R4=2-ethylhexyl.

The acrylic acid derivatives according to the present invention of formula (c) are used in a quantity from 0.01 to 5.0 wt %, based on the total composition. Quantities from 0.01 to 3 wt % are preferred, from 0.01 to 2.0 wt % are particularly preferred, and from 0.05 to 2.0 wt % are highly preferred.

The present active substance combination according to the present invention can be formulated as a rinse-off application or as a leave-on application. Application as a leave-on application is preferred. Within the leave-on applications, a formulation as a non-aerosol spray is particularly preferred.

In many cases the agents contain at least one surface-active substance, both anionic and zwitterionic, ampholytic, nonionic, and cationic surface-active substances being suitable in principle. Selection of the surface-active substances is based on the nature of the agent. In the case of a shampoo, in particular at least one surfactant from the group of the anionic, zwitterionic, or nonionic surface-active substances is selected. It is preferred in this context that at least one anionic and at least one zwitterionic surface-active substance be selected. Particularly preferably in this context, these surface-active substances are selected from the group of the particularly mild surface-active substances. In many cases, however, it has proven advantageous to select the surfactants from anionic, zwitterionic, or nonionic surfactants. The ratio between anionic and zwitterionic surface-active substances is in this context between 10:1 and 1:5. A ratio from 5:1 to 1:2 is particularly preferred.

All anionic surface-active substances suitable for use on the human body are suitable as anionic surfactants (Tanion) in preparations according to the present invention.

The following anionic surfactants have proven to be mild to particularly mild, and are particularly preferred according to the present invention:
acyl lactylates,
hydroxy mixed ether sulfates,
ether carboxylic acids of the formula R—O—(CH₂—CH₂O)ₓ—CH₂—COOH, in which R is a linear alkyl group having 8 to 30 carbon atoms and x=0 or is 1 to 16, and salts thereof,
acyl sarcosides having 8 to 24 carbon atoms in the acyl group,
acyl taurides having 8 to 24 carbon atoms in the acyl group,
acyl isethionates having 8 to 24 carbon atoms in the acyl group,
sulfosuccinic acid mono- and dialkyl esters having 8 to 24 carbon atoms in the alkyl group and sulfosuccinic acid monoalkylpolyoxyethyl esters having 8 to 24 carbon atoms in the alkyl group and 1 to 6 oxyethyl groups,
alkylpolyglycol ether sulfates of the formula R—O(CH₂—CH₂O)ₓ—OSO₃H, in which R is a preferably linear alkyl group having 8 to 30 carbon atoms and x=0 or is 1 to 12,
esters of tartaric acid and citric acid with alcohols that represent addition products of approximately 2 to 15 molecules of ethylene oxide and/or propylene oxide with fatty alcohols having 8 to 22 carbon atoms,
alkyl and/or alkenyl ether phosphates of the formula

R¹(OCH₂CH₂)ₙ—O—(PO—OX)—OR²,
in which R¹ preferably denotes an aliphatic hydrocarbon residue having 8 to 30 carbon atoms, R² denotes hydrogen, a (CH₂CH₂O)ₙR² residue, or X, n denotes numbers from 1 to 10, and X denotes hydrogen, an alkali or alkaline-earth metal, or NR³R⁴R⁵R⁶, where R³ to R⁶ mutually independently denote hydrogen or a C₁ to C₄ hydrocarbon residue,
monoglyceride sulfates and monoglyceride ether sulfates of the formula ROC—(OCH₂CH₂)ₓ—OCH₂—[CHO(CH₂CH₂O)_{y}H]—CH₂O(CH₂CH₂O)_{z}—SO₃X,
in which RCO denotes a linear or branched acyl residue having 6 to 22 carbon atoms, x, y, and z in total denote 0 or numbers from 1 to 30, preferably 2 to 10, and X denotes an alkali or alkaline-earth metal. Typical examples of monoglyceride (ether) sulfates suitable for purposes of the invention are the reaction products of lauric acid monoglyceride, coconut fatty acid monoglyceride, palmitic acid monoglyceride, stearic acid monoglyceride, oleic acid monoglyceride, and tallow fatty acid monoglyceride, and their ethylene oxide adducts, with sulfur trioxide or chlorosulfonic acid in the form of their sodium salts. It is preferable to use monoglyceride sulfates in which RCO denotes a linear acyl residue having 8 to 18 carbon atoms,
amide ether carboxylic acids, R¹—CO—NR²—CH₂CH₂—O—(CH₂CH₂O)ₙCH₂COOM, where R¹ is a straight-chain or branched alkyl or alkenyl residue having a number of carbon atoms in the chain from 2 to 30, n denotes a whole number from 1 to 20, and R² denotes hydrogen, a methyl, ethyl, propyl, isopropyl, n-butyl, tert-butyl, or isobutyl residue, and M denotes hydrogen or a metal such as an alkali metal, in particular sodium, potassium, lithium, an alkaline-earth metal, in particular magnesium, calcium, zinc, or an ammonium ion, such as ⁺NR³R⁴R⁵R⁶, where R³ to R⁶ mutually independently denote hydrogen or a C1 to C4 hydrocarbon residue. Products of this kind are obtainable, for example, from the Chem-Y company under the product designation Akypo®.
condensation products of a water-soluble salt of a water-soluble protein hydrolysate with a suitable fatty acid derivative, for example a C8 to C30 fatty acid halide. Such products have been commercially obtainable for some time under the trademarks Lamepon®, Maypon®, Gluadin®, Hostapon® KCG, or Amisoft®,
acyl glutamates, and
acyl aspartates.

It is further preferred in the case of mild anionic surfactants having polyglycol ether units that the number of glycol ether groups be equal to 1 to 20, preferably 2 to 15, particularly preferably 2 to 12. Particularly mild anionic surfactants having polyglycol ether groups without a restricted homolog distribution can also be obtained, for example, if on the one hand the number of polyglycol ether groups is equal to 4 to 12, and Zn or Mg ions are selected as a counter ion. One example thereof is the commercial product Texapon® ASV.

“Zwitterionic surfactants” (Tzwitter) refers to those surface-active compounds that carry in the molecule at least one quaternary ammonium group and at least one —COO⁽⁻⁾ or SO₃⁽⁻⁾ group. Particularly suitable zwitterionic surfactants are the so-called betaines, such as the N-alkyl-N,N-dimethylammonium glycinates, for example cocalkyldimethylammonium glycinate, N-acylaminopropyl-N,N-dimethylammonium glycinates, for example cocacylaminopropyldimethylammonium glycinate, and 2-alkyl-3-carboxymethyl-3-hydroxyethylimidazolines, having in each case 8 to 18 carbon atoms in the alkyl or acyl group, as well as cocacylaminoethylhydroxyethylcarboxymethyl glycinate. A preferred zwitterionic surfactant is the fatty acid amide derivative known by the INCI name Cocamidopropyl Betaine.

“Ampholytic surfactants” (Tampho) are understood as those surface-active compounds that contain, in addition to a C₈ to C₂₄ alkyl or acyl group in the molecule, at least one free amino group and at least one —COOH or —SO₃H group, and are capable of forming internal salts. Examples of suitable ampholytic surfactants are N-alkyl glycines, N-alkylpropionic acids, N-alkylaminobutyric acids, N-alkyliminodipropionic acids, N-hydroxyethyl-N-alkylamidopropyl glycines, N-alkyl taurines, N-alkyl sarcosines, 2-alkylaminopropionic acids, and alkylaminoacetic acids, having in each case approximately 8 to 24 carbon atoms in the alkyl group. Typical examples of amphoteric or zwitterionic surfactants are alkyl betaines, alkylamidobetaines, aminopropionates, aminoglycinates, imidazolinium betaines, and sulfobetaines.

Particularly preferred ampholytic surfactants are N-cocalkylaminopropionate, cocacylaminoethylaminopropionate, and C₁₂ to C₁₈ acyl sarcosine.

Nonionic surfactants are, for example:
addition products of 2 to 50 mol ethylene oxide and/or 0 to 5 mol propylene oxide with linear and branched fatty alcohols having 6 to 30 carbon atoms, the fatty alcohol polyglycol ethers or fatty alcohol polypropylene glycol ethers, and/or mixed fatty alcohol polyethers,
addition products of 2 to 50 mol ethylene oxide and/or 0 to 5 mol propylene oxide with linear and branched fatty acids having 6 to 30 carbon atoms, the fatty acid polyglycol ethers and/or fatty acid polypropylene glycol ethers, and/or mixed fatty acid polyethers,
addition products of 2 to 50 mol ethylene oxide and/or 0 to 5 mol propylene oxide with linear and branched alkylphenols having 8 to 15 carbon atoms in the alkyl group, the alkylphenol polyglycol ethers and/or alkylphenol polypropylene glycol ethers, and/or mixed alkylphenol polyethers,
addition products, end-capped with a methyl or C₂ to C₆ alkyl group, of 2 to 50 mol ethylene oxide and/or 0 to 5 mol propylene oxide with linear and branched fatty alcohols having 8 to 30 carbon atoms, with fatty acids having 8 to 30 carbon atoms, and with alkylphenols having 8 to 15 carbon atoms in the alkyl group, such as, for example, the grades obtainable under the marketing designations Dehydrol® LS, Dehydrol® LT (Cognis),
C₁₂ to C₃₀ fatty acid mono- and diesters of addition products of 1 to 30 mol ethylene oxide with glycerol,
addition products of 5 to 60 mol ethylene oxide with castor oil and hardened castor oil,
polyol fatty acid esters such as, for example, the commercial product Hydagen® HSP (Cognis), or Sovermol® grades (Cognis),
alkoxylated triglycerides,
alkoxylated fatty acid alkyl esters of formula (Tnio-I):

R¹CO—(OCH₂CHR²)_{w}OR³ (Tnio-I),
in which R¹CO denotes a linear or branched, saturated and/or unsaturated acyl residue having 6 to 22 carbon atoms, R² denotes hydrogen or methyl, R³ denotes linear or branched alkyl residues having 1 to 4 carbon atoms, and w denotes numbers from 1 to 20,
amine oxides,
hydroxy mixed ethers,
sorbitan fatty acid esters and addition products of ethylene oxide with sorbitan fatty acid esters, for example the polysorbates,
sugar fatty acid esters and addition products of ethylene oxide with sugar fatty acid esters,
addition products of ethylene oxide with fatty acid alkanolamides and fatty amines,
sugar surfactants of the alkyl and alkenyl oligoglycoside types, according to formula (E4-II)

R⁴O-[G]ₚ (Tnio-2)
in which R⁴ denotes an alkyl or alkenyl residue having 4 to 22 carbon atoms, G denotes a sugar residue having 5 or 6 carbon atoms, and p denotes numbers from 1 to 10.
sugar surfactants of the fatty acid N-alkylpolyhydroxyalkylamide types, a nonionic surfactant of formula (Tnio-3)

R⁵O—NR⁶—[Z] (Tnio-3)
in which R⁵CO denotes an aliphatic acyl residue having 6 to 22 carbon atoms, R⁶ denotes hydrogen, an alkyl or hydroxyalkyl residue having 1 to 4 carbon atoms, and [Z] denotes a linear or branched polyhydroxyalkyl residue having 3 to 12 carbon atoms and 3 to 10 hydroxyl groups.

A very particularly preferred embodiment contains at least one cationic surfactant.

Cationic surfactants (Tkat) are derived in general from ammonium ions and possess a structure

(NR¹R²R³R⁴)⁺A⁻ (Tkat1)

with a correspondingly negatively charged counter ion A.

In all cationic surfactants according to the present invention, the anion A is selected from the physiologically acceptable anions. Examples of the anion that may be recited are the halides, fluoride, chloride, bromide, iodide, phosphate, dihydrogen phosphate, hydrogen phosphate, hydrogen sulfate, sulfate (methosulfate) of the general formula RSO₃⁻, in which R has the meaning of saturated or unsaturated alkyl residues having 1 to 4 carbon atoms, in particular methosulfate and ethosulfate, or anionic residues of organic acids such as maleate, fumarate, oxalate, tartrate, citrate, lactate, or acetate. Particularly preferred cationic surfactants have chloride or methosulfate as an anion, with a methyl or ethyl group as residue R.

Cationic ammonium compounds of this kind of formula (Tkat1) are very well known to one skilled in the art. Residues R1 to R4 can signify, mutually independently in each case, straight-chain, branched, cyclic, aromatic saturated or unsaturated alkyl and/or alkenyl chains having at least 1 to 30 carbon atoms, hydrogen, —OH, or hydroxyethyl.

Cationic structures preferred according to the present invention are those in which residues R1, R2, and R3 each signify a methyl group and R4 represents a saturated, branched or unbranched alkyl residue having a chain length from 16 to 24 carbon atoms, and furthermore the residue R4 represents a saturated, branched or unbranched alkyl residue, very particularly preferably unbranched alkyl residue having a chain length form 18 to 24, highly preferably having a chain length from 22 to 24 carbon atoms. Preferred quaternary ammonium compounds of formula (Tkat1-1) are ammonium halides, in particular chlorides and bromides, such as alkyltrimethylammonium chlorides, dialkyldimethylammonium chlorides, and trialkylmethylammonium chlorides, e.g. distearyldimethylammonium chloride, lauryldimethylammonium chloride, lauryldimethylbenzylammonium chloride, and tricetylmethylammonium chloride, cetyltrimethylammonium chloride, cetyltrimethylammonium bromide, cetyltrimethylammonium methosulfate, stearyltrimethylammonium chloride, behenyltrimethylammonium chloride, behenyltrimethylammonium bromide, and behenyltrimethylammonium methosulfate. Cetyltrimethylammonium salts and behenyltrimethylammonium salts are preferred. The latter are particularly preferred in the form of the methosulfates and bromides. Cetyltrimethylammonium methosulfate and behenyltrimethylammonium methosulfate are the most preferred, and behenyltrimethylammonium methosulfate is highly preferred.

In an embodiment, esterquats in accordance with formula (Tkat1-2) can furthermore be used.

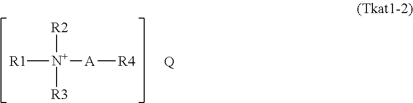

Residues R1, R2, and R3 therein are each mutually independent and can be identical or different. Residues R1, R2, and R3 signify:
a branched or unbranched alkyl residue having 1 to 4 carbon atoms, which can contain at least one hydroxyl group, for example methyl, ethyl, propyl, isopropyl, butyl, isobutyl, tert-butyl, hydroxyethyl, hydroxymethyl, or
a saturated or unsaturated, branched or unbranched, or cyclic saturated or unsaturated alkyl residue having 6 to 30 carbon atoms, which can contain at least one hydroxyl group, or
an aryl or alkaryl residue, for example phenyl or benzyl,
the residue (-A-R4), provided that at most two of residues R1, R2, or R3 can denote this residue.

The residue -(A-R4) is contained at least 1 to 3 times.

In this, A denotes:
1) —(CH₂)n-, where n=1 to 20, by preference n=1 to 10, and particularly preferably n=1 to 5, or
2) —(CH₂—CHR⁵—O)n-, where n=1 to 200, by preference 1 to 100, particularly preferably 1 to 50, and particularly preferably 1 to 20, where R5 has the meaning of hydrogen, methyl, or ethyl,
   and R4 denotes:
3) R6-O—CO—, in which R6 is a saturated or unsaturated, branched or unbranched, or a cyclic saturated or unsaturated alkyl residue having 6 to 30 carbon atoms, which can contain at least one hydroxy group, and which optionally can be further oxyethylated with 1 to 100 ethylene oxide units and/or 1 to 100 propylene oxide units, or
4) R7-CO—, in which R7 is a saturated or unsaturated, branched or unbranched, or a cyclic saturated or unsaturated alkyl residue having 6 to 30 carbon atoms, which can contain at least one hydroxy group, and which optionally can be further oxyethylated with 1 to 100 ethylene oxide units and/or 1 to 100 propylene oxide units, and
   Q denotes a physiologically acceptable organic or inorganic anion that has been described as above under formula (Tkat1). Preferred esterquats are quaternized ester salts of fatty acids with triethanolamine, quaternized ester salts of fatty acids with diethanolalkylamines, and quaternized ester salts of fatty acids with 1,2-dihydroxypropyldialkylamines. Such products are marketed, for example, under the trademarks Rewoquat®, Stepantex®, Dehyquart®, and Armocare®. Examples of such esterquats are the products Armocare® VGH-70—an N,N-bis(2-palmitoyloxyethyedimethylammonium chloride—as well as Dehyquart® F-75, Dehyquart® C-4046, Dehyquart® L-80, Dehyquart® F-30, Dehyquart® AU-35, Rewoquat® WE18, Rewoquat® WE38 DPG, and Stepantex® GS 90.

Further compounds of formula (Tkat1-2) particularly preferred according to the present invention conform to formula (Tkat1-2.1), the cationic betaine esters:

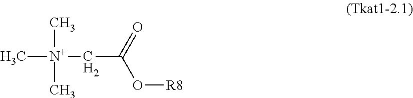

The meaning of R8 corresponds to that of R7.

Further preferred cationic surfactants are cationic surfactants of formula (Tkat-2)

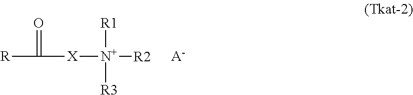

R therein denotes a substituted or unsubstituted, branched or straight-chain alkyl or alkenyl residue having 11 to 35 carbon atoms in the chain,
X denotes —O— or —NR5-,
R¹ denotes an alkylene group, having 2 to 6 carbon atoms, which can be substituted or unsubstituted; in the event of a substitution, substitution with an —OH or —NH group is preferred,
R², R³ denote, mutually independently in each case, an alkyl or hydroxyalkyl group having 1 to 6 carbon atoms in the chain, such that the chain can be straight or branched. Examples of residues according to the present invention are methyl, ethyl, propyl, isopropyl, butyl, isobutyl, tert-butyl, pentyl, isopentyl, neopentyl, hexyl, isohexyl, hydroxyalkyl, dihydroxyalkyl, hydroxyethyl, hydroxypropyl, dihydroxypropyl, hydroxybutyl, dihydroxybutyl, trihydroxybutyl, trihydroxypropyl, dihydroxyethyl, R5 denotes hydrogen or a C1 to C6 straight-chain or branched alkyl or alkenyl residue, which can also be substituted with a hydroxy group, in particular methyl, ethyl, propyl, isopropyl, butyl, isobutyl, tert-butyl, pentyl, isopentyl, neopentyl, hexyl, isohexyl, hydroxyethyl, hydroxypropyl, dihydroxypropyl, hydroxybutyl, dihydroxybutyl, trihydroxybutyl, trihydroxypropyl, dihydroxyethyl, and
A⁻ denotes an anion as already described with reference to formula (Tkat1).

Within this structure class, the compounds having one of the following structures are used in preferred fashion:

CH₃(CH₂)₂₀CONH(CH₂)₃—N⁺(CH₃)₂—CH₂CH₃A- (Tkat-3)

CH₃(CH₂)₂₀CONH(CH₂)₃—N⁺(CH₃)₂—CH₂(CHOH)CH₂OHA- (Tkat-4)

CH₃(CH₂)₂₀COOCH₂CHOHCH₂—N⁺(CH₃)₃A- (Tkat-5)

CH₃(CH₂)₂₀CONH(CH₂)₃—N⁺(CH₃)₂—CH₂CH₂OHA- (Tkat-6).

Examples of commercial products of this kind are Schercoquat BAS, Lexquat AMG-BEO, Akypoquat 131, or Incroquat Behenyl HE.

In a particularly preferred embodiment of the invention, the agents according to the present invention further contain at least one amine and/or cationized amine, in particular an amidoamine and/or cationized amidoamine, having the following structural formulas:

R¹—NH—(CH₂)ₙ—NR²R³ (Tkat7) and/or

R¹—NH—(CH₂)ₙ—NR²R³R⁴ (Tkat8)

in which R1 denotes an acyl or alkyl residue having 6 to 30 carbon atoms, which can be branched or unbranched, saturated or unsaturated, and such that the acyl residue and/or alkyl residue can contain at least one OH group, and
R2, R3, and R4 each denote, mutually independently in each case, hydrogen or an alkyl residue having 1 to 4 carbon atoms, which can be identical or different, saturated or unsaturated, and
n signifies a whole number between 1 and 10.

The anion is again selected from the physiologically acceptable anions as already described with reference to foimula (Tkat1).

A composition in which the amine and/or quaternized amine in accordance with the general formulas (Tkat7) and/or (Tkat8) is an amidoamine and/or a quaternized amidoamine, in which R1 signifies a branched or unbranched, saturated or unsaturated acyl residue having 6 to 30 carbon atoms, which can contain at least one OH group, is preferred. A fatty acid residue made up of oils and waxes, in particular of natural oils and waxes, is preferred in this context. Lanolin, beeswax, or candelilla wax are appropriate examples thereof. Also preferred are those amidoamines ad/or quaternized amidoamines in which R2, R3, and/or R4 in formulas (Tkat7) and/or (Tkat8) signify a residue in accordance with the general formula CH₂CH₂OR5, in which R5 can have the meaning of alkyl residues having 1 to 4 carbon atoms, hydroxyethyl, or hydrogen. The preferred value of n in the general formulas (Tkat7) and/or (Tkat8) is a whole number between 2 and 5.

Also preferred are amidoamines and/or quaternized amidoamines of the general formulas (Tkat7) and/or (Tkat8) in which the anion is a halide ion or a compound of the general formula RSO₃⁻, in which R has the meaning of saturated or unsaturated alkyl residues having 1 to 4 carbon atoms.

The alkylamidoamines both can be present as such, and can be converted by protonation in a correspondingly acid solution into a quaternary compound, in the composition. The cationic alkylamidoamines are preferred according to the present invention.

The following amidoamines, for example, are appropriate as amidoamines to be used according to the present invention, which optionally can be quaternized: Witcamine 100 (Witco, INCI name. Cocamidopropyl Dimethylamine), Incromine BB (Croda, INCI name: Behenamidopropyl Dimethylamine), Mackine 401 (McIntyre, INCI name: Isostearylamidopropyl Dimethylamine) and other Mackine grades, Adogen S18V (Witco, INCI name: Stearylamidopropyl Dimethylamine) and, as permanently cationic aminoamines: Rewoquat RTM 50 (Witco Surfactants GmbH, INCI name: Ricinoleamidopropyltrimonium Methosulfate), Empigen CSC (Albright & Wilson, INCI name: Cocamidopropyltrimonium Chloride), Swanol Lanoquat DES-50 (Nikko, INCI name: Quatemium-33), Rewoquat UTM 50 (Witco Surfactants GmbH, Undecyleneamidopropyltrimonium Methosulfate).

Lastly, quaternary imidazoline compounds can be used as cationic surfactants, i.e. compounds that comprise a positively charged imidazoline ring. Formula (Tkat9) depicted below shows the structure of these compounds:

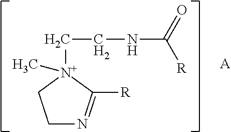

Residues R denote, mutually independently in each case, a saturated or unsaturated, linear or branched hydrocarbon residue having a chain length from 8 to 30 carbon atoms. The preferred compounds of formula (Tkat9) each contain the same hydrocarbon residue for R. Residues R denote, mutually independently in each case, a saturated or unsaturated, linear or branched hydrocarbon residue having a chain length from 8 to 18 carbon atoms. The preferred compounds of formula I each contain the same hydrocarbon residue for R. The chain length of residues R is preferably 12 to 18 carbon atoms. Compounds having a chain length of at least 16 carbon atoms are particularly preferred, and those having at least 18 carbon atoms are very particularly preferred. The chain length of residues R is highly preferably at least 20 carbon atoms. Compounds having a chain length of at least 21 carbon atoms are most preferred. Preferred residues R are cetyl, oleyl, palmityl, stearyl, and behenyl. “A” signifies a physiologically acceptable anion as already described with reference to the cationic compounds of formula (Tkat1). Examples that are particularly in accordance with the present invention are obtainable, for example, under the INCI names Quaternium-27, Quatemium-72, and Quatemium-83. A highly preferred commercial product of this chain length is known, for example, by the name Quatemium-91.

The teaching of the present invention of course also encompasses the use of at least two cationic surfactants. In this case it is preferred if the cationic surfactants are selected from two different structure classes.

The cationic surfactants are contained in the compositions according to the present invention in quantities from 0.01 to 20 wt %, preferably in quantities from 0.01 to 10 wt %, and very particularly preferably in quantities from 0.1 to 7.5 wt %. The best results of all are obtained with quantities from 0.1 to 5 wt %, based in each case on the total composition of the respective agent.

Cationic, zwitterionic, and/or amphoteric surfactants, as well as mixtures thereof, can be preferred according to the present invention.

The surfactants (T) are used in quantities from 0.05 to 45 wt %, preferably 0.1 to 30 wt %, and very particularly preferably from 0.5 to 25 wt %, based on the total agent utilized according to the present invention.

Emulsifier agents usable according to the present invention are, for example:
ether carboxylic acids of the formula R—O—(CH₂—CH₂O)ₓ—CH₂—COOH, in which R is a linear alkyl group having 8 to 30 carbon atoms and x=0 or is 1 to 16,
acyl sarcosides having 8 to 24 carbon atoms in the acyl group,
acyl taurides having 8 to 24 carbon atoms in the acyl group,
acyl isethionates having 8 to 24 carbon atoms in the acyl group,
sulfosuccinic acid mono- and -dialkyl esters having 8 to 24 carbon atoms in the alkyl group and sulfosuccinic acid monoalkylpolyoxyethyl esters having 8 to 24 carbon atoms in the alkyl group and 1 to 6 oxyethyl groups,
esters of tartaric acid and citric acid with alcohols representing addition products of approximately 2 to 15 molecules of ethylene oxide and/or propylene oxide with fatty alcohols having 8 to 22 carbon atoms,
alkyl and/or alkenyl ether phosphates as already described in the context of the surfactants,
monoglyceride sulfates and monoglyceride ether sulfates as already described in the context of the surfactants,
amide ether carboxylic acids as described in the section about surfactants;
condensation products of a water-soluble salt of a water-soluble protein hydrolysate-fatty acid condensation product,
zwitterionic surfactants (Tzwitter),
ampholytic surfactants (Tampho),
sugar surfactants of the alkyl or alkenyl oligoglycoside type,
sugar surfactants of the fatty acid-N-alkylpolyhydroxyalkylamide type,
addition products of 5 to 60 mol ethylene oxide with castor oil and hardened castor oil,
polyol fatty acid esters, for example the commercial product Hydagen® HSP (Cognis) or Sovermol® grades (Cognis),
amine oxides,
hydroxy mixed ethers, for example of the formula R¹O[CH₂CH(CH₃)O]ₓ(CH₂CHR²O)_{y}[CH₂CH(OH)R³]_{Z} where R¹ denotes a linear or branched, saturated or unsaturated alkyl and/or alkenyl residue having 2 to 30 carbon atoms, R² denotes hydrogen, a methyl, ethyl, propyl, or isopropyl residue, R³ denotes a linear or branched alkyl residue having 2 to 30 carbon atoms, x denotes 0 or a number from 1 to 20, Y denotes a number from 1 to 30, and z denotes the number 1, 2, 3, 4 or 5,
sorbitan fatty acid esters and addition products of ethylene oxide with sorbitan fatty acid esters, such as e.g. the polysorbates,
sugar fatty acid esters and addition products of ethylene oxide with sugar fatty acid esters,
addition products of ethylene oxide with fatty acid alkanolamides and fatty amines,
sugar surfactants of the alkyl and alkenyl oligoglycoside types in accordance with formula (E4-II),
esterquats,
alkylamidoamines and quaternized alkylamidoamines,
C₈ to C₂₂ alkyl mono- and oligoglycosides and ethoxylated analogs thereof, where degrees of oligomerization from 1.1 to 5, in particular 1.2 to 2.0, and glucose as a sugar component, are preferred,
mixtures of alkyl(oligo)glucosides and fatty alcohols, for example the commercially obtainable product Montanov® 68,
addition products of 5 to 60 mol ethylene oxide with castor oil and hardened castor oil,
partial esters of polyols having 3 to 6 carbon atoms with saturated fatty acids having 8 to 22 carbon atoms,
sterols.
Phospholipids. These are understood chiefly as the glucose phospholipids, which are obtained e.g. as lecithins or phosphatidylcholines from, for example, egg yolk or plant seeds (e.g. soybeans).
Fatty acid esters of sugars and sugar alcohols, such as sorbitol,
polyglycerols and polyglycerol derivatives such as e.g. polyglycerol-12-hydroxystearate (commercial product Dehymuls® PGPH).

Very particularly, cationic and/or amphoteric and/or zwitterionic polymers are used as further ingredients in the agents according to the present invention.

Some examples of particularly preferred polymers are described below.

The cationic polymers can be homo- or copolymers, the quaternary nitrogen groups being contained either in the polymer chain or, by preference, as a substituent on one or more of the monomers. The ammonium-group-containing monomers can be copolymerized with non-cationic monomers. Suitable cationic monomers are unsaturated, radically polymerizable compounds that carry at least one cationic group, in particular ammonium-substituted vinyl monomers such as, for example, trialkylmethacryloxyalkylammonium, trialkylacryloxyalkylammonium, dialkyldiallylammonium, and quaternary vinylammonium monomers having cyclic groups containing cationic nitrogens, such as pyridinium, imidazolium, or quaternary pyrrolidones, e.g. alkylvinylimidazolium, alkylvinylpyridinium, or alkyvinylpyrrolidone salts. The alkyl groups of these monomers are by preference lower alkyl groups such as, for example, C1 to C7 alkyl groups, particularly preferably C1 to C3 alkyl groups.

The ammonium-group-containing monomers can be copolymerized with non-cationic monomers. Suitable comonomers are, for example, acrylamide, methacrylamide; alkyl and dialkyl acrylamide, alkyl and dialkyl methacrylamide, alkyl acrylate, alkyl methacrylate, vinylcaprolactone, vinylcaprolactam, vinylpyrrolidone, vinyl esters, e.g. vinyl acetate, vinyl alcohol, propylene glycol, or ethylene glycol, the alkyl groups of these monomers being by preference C1 to C7 alkyl groups, particularly preferably C1 to C3 alkyl groups.

Suitable polymers having quaternary amine groups are, for example, the polymers described in the CTFA Cosmetic Ingredient Dictionary under the “Polyquaternium” designations, such as methylvinylimidazolium chloride/vinylpyrrolidone copolymer (Polyquatemium-16), or quaternized vinylpyrrolidone/dimethylaminoethyl methacrylate copolymer (Polyquatemium-11).

Suitable among the cationic polymers that can be contained in the agent according to the present invention is, for example, the vinylpyrrolidone/dimethylaminoethyl methacrylate methosulfate copolymer that is marketed under the commercial names Gafquat® 755 N and Gafquat® 734 by the GAF company, USA, and of which Gafquat® 734 is particularly suitable. Further cationic polymers are, for example, the copolymer of polyvinylpyrrolidone and imidazolimine methochloride marketed by BASF, Germany, under the trade name Luviquat® HM 550, the terpolymer of dimethyldiallylammonium chloride, sodium acrylate, and acrylamide marketed by the Calgon company, USA, under the trade name Merquat® Plus 3300, and the vinylpyrrolidone/methacrylamidopropyltrimethylammonium chloride copolymer marketed by the ISP company under the trade name Gafquat® HS 100.

A particularly suitable homopolymer is the poly(methacryloyloxyethyltrimethylammonium) chloride (crosslinked, if desired) having the INCI name Polyquaternium-37. Such products are available commercially, for example, under the designations Rheocare® CTH (Cosmetic Rheologies) and Synthalen® CR (3V Sigma). Further polymer dispersions are obtainable commercially under the designations Salcare® SC 95 (approx. 50% polymer proportion, further components: mineral oil (INCI name: Mineral Oil) and tridecylpolyoxypropylenepolyoxyethylene ether (INCI name: PPG-1-Trideceth-6)), and Salcare® SC 96 (approx. 50% polymer proportion, further components: mixture of diesters of propylene glycol with a mixture of caprylic and capric acid (INCI name: Propylene Glycol Dicaprylate/Dicaprate) and tridecylpolyoxypropylenepolyoxyethylene ether (INCI name: PPG-1-Trideceth-6)).

A copolymer preferred according to the present invention is the crosslinked acrylamide-methacryloyloxyethyltrimethylammonium chloride copolymer. Such copolymers, in which the monomers are present at a weight ratio of approximately 20:80, are obtainable commercially as an approximately 50% nonaqueous polymer dispersion under the name Salcare® SC 92.

Suitable cationic polymers that are derived from natural polymers are cationic derivatives of polysaccharides, for example cationic derivatives of cellulose, starch, or guar. Chitosan and chitosan derivatives are also suitable.

A cationic cellulose is marketed by Amerchol under the designation Polymer JR® 400 and has the INCI name Polyquatemium-10. A further cationic cellulose bears the INCI name Polyquaternium-24 and is marketed by Amerchol under the trade name Polymer LM-200. Further commercial products are the compounds Celquat® H 100 and Celquat® L 200. The aforesaid commercial products are preferred cationic celluloses.

Suitable cationic guar derivatives are marketed under the commercial designation Jaguar® and have the INCI name Guar Hydroxypropyltrimonium Chloride. Particularly suitable cationic guar derivatives are additionally available commercially from the Hercules company under the designation N-Hance®. Further cationic guar derivatives are marketed by the Cognis company under the designation Cosmedia®. A preferred cationic guar derivative is the commercial product AquaCat® of the Hercules company. This raw material is a cationic guar derivative that is already predissolved.

A suitable chitosan is marketed, for example, by the Kyowa Oil & Fat company, Japan, under the trade name Flonac®. A preferred chitosan salt is chitosonium pyrrolidonecarboxylate, which is marketed e.g. under the designation Kytamer® PC by the Amerchol company, USA. Further chitosan derivatives are readily available commercially under the commercial designations Hydagen® CMF, Hydagen® HCMF, and Chitolam® NB/101.

Further preferred cationic polymers are, for example:
cationic alkyl polyglycosides,
cationized honey, for example the commercial product Honeyquat® 50,
polymeric dimethyldiallylammonium salts and copolymers thereof with esters and amides of acrylic acid and methacrylic acid. The products obtainable commercially under the designations Merquat® 100 (poly(dimethyldiallylammonium chloride)) and Merquat® 550 (dimethyldiallylammonium chloride/acrylamide copolymer) are examples of such cationic polymers,
vinylpyrrolidone/vinylimidazolium methochloride copolymers, such as those offered under the designations Luviquat® FC 370, FC 550, FC 905, and HM 552,
quaternized poly(vinylalcohol),
and the polymers known under the names Polyquatemium-2, Polyquatemium-17, Polyquaternium-18, and Polyquaternium-27, having quaternary nitrogen atoms in the main polymer chain,
vinylpyrrolidone/vinylcaprolactam/acrylate terpolymers such as those having acrylic acid esters and acrylic acid amides as a third monomer module, and offered commercially e.g. under the designation Aquaflex® SF 40.

Also usable according to the present invention are the copolymers of vinylpyrrolidone such as those obtainable as the commercial products Copolymer 845 (manufacturer: ISP), Gaffix® VC 713 (manufacturer: ISP), Gafquat® ASCP 1011, Gafquat® HS 110, Luviquat® 8155, and Luviquat® MS 370.

Cationized protein hydrolysates are further to be included among the cationic polymers, in which context the underlying protein hydrolysate can derive from animals, for example from collagen, milk, or keratin, from plants, for example from wheat, corn, rice, potatoes, soy, or almonds, from marine life forms, for example from fish collagen or algae, or from biotechnologically obtained protein hydrolysates. Typical examples that may be recited of cationic protein hydrolysates and derivatives according to the present invention are the products listed under the INCI names in the “International Cosmetic Ingredient Dictionary and Handbook” (seventh edition 1997, The Cosmetic, Toiletry, and Fragrance Association, 1101 17th Street, N.W., Suite 300, Washington, D.C. 20036-4702), and available commercially.

The cationic polymers are contained in the compositions according to the present invention preferably in quantities from 0.05 to 10 wt %, based on the total agent. Quantities from 0.1 to 5 wt % are particularly preferred.

Amphoteric polymers, like the cationic polymers, are very particularly preferred polymers. Particularly preferred amphoteric polymers are copolymers of diallyldimethylammonium chloride and acrylic acid. These copolymerizates are marketed under the INCI name Polyquaternium-22, inter alia with the trade name Merquat® 280 (Nalco). Amphoteric polymers used very particularly preferably according to the present invention are terpolymers of diallyldimethylammonium chloride, acrylamide, and acrylic acid. These copolymerizates are marketed under the INCI name Polyquaternium-39, inter alia with the commercial name Merquat® Plus 3330 (Nalco).

The amphoteric polymers are contained in the agents according to the present invention preferably in quantities from 0.05 to 10 wt %, based on the total agent. Quantities from 0.1 to 5 wt % are particularly preferred.

Anionic polymers are anionic polymers that comprise carboxylate and/or sulfonate groups. Examples of anionic monomers of which such polymers can be made up are acrylic acid, methacrylic acid, crotonic acid, maleic acid anhydride, and 2-acrylamido-2-methylpropanesulfonic acid. The acid groups can be present in this context entirely or partially as a sodium, potassium, ammonium, mono- or triethanolammonium salt.

Anionic polymers that contain 2-acrylamido-2-methylpropanesulfonic acid as the only monomer or co-monomer have proven to be very particularly effective, in which context the sulfonic acid group can be present entirely or partly as a sodium, potassium, ammonium, or mono- or triethanolammonium salt. The homopolymer of 2-acrylamido-2-methylpropanesulfonic acid that is obtainable commercially, for example, under the designation Rheothik® 11-80 is particularly preferred.

Preferred anionic polymers are acrylic acid/acrylamide copolymers and in particular polyacrylamide copolymers with sulfonic acid group-containing monomers. A particularly preferred anionic setting polymer is made up of 70 to 55 mol % acrylamide and 30 to 45 mol % 2-acrylamido-2-methylpropanesulfonic acid, the sulfonic acid group being present entirely or partially as a sodium, potassium, ammonium, mono-, or triethanolammonium salt. This copolymer can also be present in crosslinked form, polyolefinically unsaturated compounds such as tetraallyoxyethane, allylsucrose, allylpentaerythritol, and methylene bisacrylamide preferably being used as crosslinking agents. One such polymer is contained in the commercial product Sepigel® 305 of the SEPPIC company. The utilization of this compound, which besides the polymer component contains a hydrocarbon mixture (C₁₃ to C₁₄ isoparaffin) and a nonionogenic emulsifier (Laureth-7), has proven particularly advantageous in the context of the teaching according to the present invention.

The sodium acryloyl dimethyl taurate copolymers marketed, under the designation Simulgel® 600, as a compound with isohexadecane and polysorbate-80 have also proven particularly effective according to the present invention.

Similarly preferred anionic homopolymers are uncrosslinked and crosslinked polyacrylic acids. Allyl ethers of pentaerythritol, of sucrose, and of propylene can be preferred crosslinking agents. Such compounds are obtainable commercially, for example, under the trademark Carbopol®.

Copolymers of maleic acid anhydride and methylvinyl ether, in particular those having crosslinks, are also color-preserving polymers. A maleic acid/methylvinyl ether copolymer crosslinked with 1,9-decadiene is obtainable commercially under the designation Stabileze® QM.

The anionic polymers are contained in the agents according to the present invention preferably in quantities from 0.05 to 10 wt %, based on the total agent. Quantities from 0.1 to 5 wt % are particularly preferred.

Suitable nonionogenic polymers are, for example:
Vinylpyrrolidone/vinyl ester copolymers such as those marketed, for example, under the trademark Luviskol® (BASF). Luviskol® VA 64 and Luviskol® VA 73, which are each vinylpyrrolidone/vinyl acetate copolymers, are likewise preferred nonionic polymers.
Cellulose ethers such as hydroxypropyl cellulose, hydroxyethyl cellulose, and methylhydroxypropyl cellulose, such as those marketed, for example, under the trademarks Culminal® and Benecel® (AQUALON) and Natrosol® grades (Hercules).
Starch and derivatives thereof, in particular starch ethers, for example Structure® XL (National Starch), a multifunctional, salt-tolerant starch,
shellac,
polyvinylpyrrolidones such as those marketed, for example, under the designation Luviskol® (BASF).

The nonionic polymers are contained in the compositions according to the present invention preferably in quantities from 0.05 to 10 wt %, based on the total agent. Quantities from 0.1 to 5 wt % are particularly preferred.

The polymers (P) are contained in the compositions used according to the present invention preferably in quantities from 0.01 to 30 wt %. Quantities from 0.01 to 25, in particular from 0.01 to 15 wt %, are particularly preferred.

With particular preference, the compositions according to the present invention contain fatty substances (Fat) as a further active substance. “Fatty substances” (Fat) are to be understood as fatty acids, fatty alcohols, natural and synthetic waxes, which can be present both in solid form and in liquid form in aqueous dispersion, and natural and synthetic cosmetic oil components.

The fatty acids (Fatac) that can be used are linear and/or branched, saturated and/or unsaturated fatty acids having 6 to 30 carbon atoms. Fatty acids having 10 to 22 carbon atoms are preferred. Among those that might be recited are, for example, isostearic acids, such as the commercial products Emersol® 871 and Emersol® 875, and isopalmitic acids such as the commercial product Edenor® IP 95, as well as all other fatty acids marketed under the Edenor® commercial designations (Cognis). Further typical examples of such fatty acids are hexanoic acid, octanoic acid, 2-ethylhexanoic acid, decanoic acid, lauric acid, isotridecanoic acid, myristic acid, palmitic acid, palmitoleic acid, stearic acid, isostearic acid, oleic acid, elaidic acid, petroselinic acid, linoleic acid, linolenic acid, elaeostearic acid, arachidic acid, gadoleic acid, behenic acid, and erucic acid, as well as industrial mixtures thereof.

The quantity used is 0.1 to 15 wt % based on the total agent. The quantity is preferably 0.5 to 10 wt %, and quantities from 1 to 5 wt % can be very particularly advantageous.

Fatty alcohols (Fatal) that can be used are saturated, mono- or polyunsaturated, branched or unbranched fatty alcohols having C₆ to C₃₀, preferably C₁₀ to C₂₂, and very particularly preferably C₁₂ to C₂₂ carbon atoms. Usable in the context of the invention are, for example, decanol, octanol, octenol, dodecenol, decenol, octadienol, dodecadienol, decadienol, oleyl alcohol, erucyl alcohol, ricinol alcohol, stearyl alcohol, isostearyl alcohol, cetyl alcohol, lauryl alcohol, myristyl alcohol, arachidyl alcohol, capryl alcohol, caprinyl alcohol, linoleyl alcohol, linolenyl alcohol, and behenyl alcohol, as well as Guerbet alcohols thereof, this listing being intended to be exemplary and not limiting in nature. Fatty alcohols can represent a mixture of different fatty alcohols. Such substances are, for example, available for purchase under the designations Stenol®, e.g. Stenol® 1618, or Lanette®, e.g. Lanette® O, or Lorol®, e.g. Lorol® C8, Lorol® C14, Lorol® C18, Lorol® C8-18, HD-Ocenol®, Crodacol®, e.g. Crodacol® CS, Novol®, Eutanol® G, Guerbitol® 16, Guerbitol® 18, Guerbitol® 20, Isofol® 12, Isofol® 16, Isofol® 24, Isofol® 36, Isocarb® 12, Isocarb® 16, or Isocarb® 24. It is of course also possible according to the present invention to use wool-wax alcohols such as those available for purchase under the designations Corona®, White Swan®, Coronet®, or Fluilan®. The fatty alcohols are used in quantities from 0.1 to 30 wt % based on the total preparation, preferably in quantities from 0.1 to 20 wt %.

Natural or synthetic waxes (Fatwax) that can be used according to the present invention are solid paraffins or isoparaffins, carnauba waxes, beeswaxes, candelilla waxes, ozocerites, ceresin, spermaceti, sunflower wax, fruit waxes such as, for example, apple wax or citrus wax, microcrystalline waxes made from PE or PP. Such waxes are obtainable, for example, via Kahl & Co., Trittau.

The quantity used is 0.1 to 50 wt % based on the total agent, preferably 0.1 to 20 wt %, and particularly preferably 0.1 to 15 wt % based on the total agent.

The total quantity of oily and fatty components in the agents according to the present invention is usually 0.5 to 75 wt %, based on the total agent. Quantities from 0.5 to 35 wt % are preferred according to the present invention.

Protein hydrolysates and/or derivatives thereof are a further synergistic active substance according to the present invention in the compositions according to the present invention having the active substance complex according to the present invention.

According to the present invention, protein hydrolysates of both vegetable and animal origin, or of marine or synthetic origin, can be used.

Animal protein hydrolysates are, for example, protein hydrolysates of elastin, collagen, keratin, silk, and milk protein, which can also be present in the form of salts. Such products are marketed, for example, under the trademarks Dehylan® (Cognis), Promois® (Interorgana), Collapuron® (Cognis), Nutrilan® (Cognis), Gelita-Sol® (Deutsche Gelatine Fabriken Stoess & Co), Lexein® (Inolex), and Kerasol® (Croda).

Also preferred according to the present invention are vegetable protein hydrolysates such as, for example, soy, almond, pea, potato, and wheat protein hydrolysates. Such products are obtainable, for example, under the trademarks Gluadin® (Cognis), DiaMin® (Diamalt), Lexein® (Inolex), Hydrosoy® (Croda), Hydrolupin® (Croda), Hydrosesame® (Croda), Hydrotritium® (Croda), and Crotein® (Croda).

Further protein hydrolysates preferred according to the present invention are of marine origin These include, for example, collagen hydrolysates from fish or algae, as well as protein hydrolysates from mussels and/or pearl hydrolysates. Examples of pearl extracts according to the present invention are the commercial products Pearl Protein Extract BG® or Crodarom® Pearl.

The protein hydrolysates (P) are contained in the compositions in concentrations from 0.001 wt % to 20 wt %, by preference from 0.05 wt % to 15 wt %, and very particularly preferably in quantities from 0.05 wt % to 5 wt %.

The effect of the compositions according to the present invention can furthermore be enhanced by a 2-pyrrolidinone-5-carboxylic acid and derivatives thereof (J). The sodium, potassium, calcium, magnesium, or ammonium salts, in which the ammonium ion carries, besides hydrogen, one to three C₁ to C₄ alkyl groups, are preferred. The sodium salt is very particularly preferred. The quantities utilized in the agents according to the present invention are by preference 0.05 to 10 wt %, based on the entire agent, particularly preferably 0.1 to 5, and in particular 0.1 to 3 wt %.

A further preferred group of ingredients of the compositions according to the present invention having the active substance complex according to the present invention is vitamins, provitamins, or vitamin precursors. Vitamins, provitamins, and vitamin precursors that are allocated to groups A, B, C, E, F, and H are particularly preferred.

The group of substances referred to as “vitamin A” includes retinol (vitamin A₁) as well as 3,4-didehydroretinol (vitamin A₂). β-Carotene is the provitamin of retinol. Vitamin A components that are suitable according to the present invention are, for example, vitamin A acid and esters thereof, vitamin A aldehyde, and vitamin A alcohol, as well as esters thereof such as the palmitate and acetate. The agents according to the present invention contain the vitamin A component preferably in quantities from 0.05 to 1 wt %, based on the total preparation.

Members of the vitamin B group or vitamin B complex are, among others:
Vitamin B₁ (thiamine)
Vitamin B₂ (riboflavin)
Vitamin B₃. The compounds nicotinic acid and nicotinic acid amide (niacinamide) are often listed under this designation. Nicotinic acid amide is preferred according to the present invention; it is contained in the agents used according to the present invention preferably in quantities from 0.05 to 1 wt % based on the total agent.
Vitamin B₅ (pantothenic acid, panthenol, and pantolactone). In the context of this group, panthenol and/or pantolactone are preferably used. Derivatives of panthenol that are usable according to the present invention are, in particular, the esters and ethers of panthenol as well as cationically derivatized panthenols. Individual representatives are, for example, panthenol triacetate, panthenol monoethyl ether and the monoacetate thereof, as well as cationic panthenol derivatives. The aforesaid compounds of the vitamin B₅ type are contained in the agents according to the present invention preferably in quantities from 0.05 to 10 wt % based on the total agent. Quantities from 0.1 to 5 wt % are particularly preferred.
Vitamin B₆ (pyridoxine as well as pyridoxamine and pyridoxal).

Vitamin C (ascorbic acid). Vitamin C is utilized in the agents according to the present invention preferably in quantities from 0.1 to 3 wt % based on the total agent. Utilization in the form of the palmitic acid ester, the glucosides, or the phosphates can be preferred. Utilization in combination with tocopherols can likewise be preferred.

Vitamin E (tocopherols, in particular α-tocopherol). Tocopherol and its derivatives, which include in particular the esters such as the acetate, the nicotinate, the phosphate, and the succinate, are contained in the agents according to the present invention preferably in quantities from 0.05 to 1 wt % based on the total agent.

Vitamin F. The term “vitamin F” is usually understood to mean essential fatty acids, in particular linoleic acid, linolenic acid, and arachidonic acid.

Vitamin H. “Vitamin H” refers to the compound (3aS,4S,6aR)-2-oxohexahydrothienol[3,4-d]-imidazole-4-valeric acid, for which the trivial name “biotin” has, however, now become established. Biotin is contained in the agents according to the present invention preferably in quantities from 0.0001 to 1.0 wt %, in particular in quantities from 0.001 to 0.01 wt %.

The compositions according to the present invention preferably contain vitamins, provitamins, and vitamin precursors from groups A, B, E, and H. Panthenol, pantolactone, pyridoxine and its derivatives, as well as nicotinic acid amide and biotin, are particularly preferred.

Lastly, the use of plant extracts (L) in the compositions according to the present invention yields further advantages. According to the present invention the extracts from green tea, oak bark, nettle, witch hazel, hops, henna, chamomile, burdock root, horsetail, hawthorn, linden blossoms, almond, aloe vera, pine needles, horse chestnut, sandalwood, juniper, coconut, mango, apricot, lemon, wheat, kiwi fruit, melon, orange, grapefruit, salvia, rosemary, birch, mallow, valerian, lady's-smock, wild thyme, yarrow, thyme, lemon balm, restharrow, coltsfoot, hibiscus, meristem, ginseng, coffee, cocoa, moring a, and ginger root are especially preferred. It can be highly preferred according to the present invention if so-called Ayurvedic plant extracts are used as plant extracts. Included among the traditional Ayurvedic plants are Aegle marmelos (bilwa), Cyperus rotundus (nagar motha), Emblica officinalis (amalki), Morida citrifolia (ashyuka), Tinospora cordifolia (guduchi), Santalum album (chandana), Crocus sativus (kumkuma), Cinnamonum zeylanicum, and Nelumbo nucifera (kamala).

As a further essential ingredient, the agents according to the present invention can contain purine and/or (a) derivative(s) of purine. Preferred compositions according to the present invention contain purine and/or purine derivatives in narrower quantity ranges. Cosmetic agents preferred according to the present invention are characterized here in that they contain, based on their weight, 0.001 to 2.5 wt %, by preference 0.0025 to 1 wt %, particularly preferably 0.005 to 0.5 wt %, and in particular 0.01 to 0.1 wt % purine(s) and/or (a) purine derivative(s).

The following compounds are preferred according to the present invention among purine, the purines, and the purine derivatives: purine, adenine, guanine, uric acid, hypoxanthine, 6-purinethiol, 6-thioguanine, xanthine, caffeine, theobromine, and theophylline. In hair-cosmetic formulations, caffeine has proven especially successful; it can be used, for example, in shampoos, conditioners, hair tonics, and/or lotions in quantities by preference from 0.005 to 0.25 wt %, more preferably from 0.01 to 0.1 wt %, and in particular from 0.01 to 0.05 wt % (based in each case on the composition).

A further preferred active substance for additional use in the agents according to the present invention is taurine and/or a derivative of taurine. “Taurine” is understood exclusively as 2-aminoethanesulfonic acid as well as explicitly recited derivatives of taurine. “Derivatives of taurine” are understood as N-monomethyltaurine and N,N-dimethyltaurine. Also understood as further taurine derivatives are taurines that occur naturally as metabolic products in plant and/or animal and/or marine organisms. These include, for example, although not preferably, breakdown products of cysteine, in particular cysteine sulfinic acid. Further taurine derivatives for purposes of the present invention are taurocholic acid and hypotaurine.

Agents according to the present invention that contain, based on their weight, 0.0001 to 10.0 wt %, by preference 0.0005 to 5.0 wt %, particularly preferably 0.001 to 2.0 wt %, and in particular 0.001 to 1.0 wt % taurine and/or a derivative of taurine are particularly preferred.

It can also prove to be advantageous if penetration adjuvants and/or swelling agents (M) are contained in the compositions according to the present invention. To be included thereamong are, for example, urea and urea derivatives, guanidine and derivatives thereof, arginine and derivatives thereof, water glass, imidazole and derivatives thereof, histidine and derivatives thereof, benzyl alcohol, glycerol, glycol and glycol ethers, propylene glycol and propylene glycol ethers, for example propylene glycol monoethyl ether, carbonates, hydrogen carbonates, diols and triols, and in particular 1,2-diols and 1,3-diols such as, for example, 1,2-propanediol, 1,2-pentanediol, 1,2-hexanediol, 1,2-dodecanediol, 1,3-propanediol, 1,6-hexanediol, 1,5-pentanediol, 1,4-butanediol.

Hydantoins can be a particularly preferred group of swelling agents. Compositions according to the present invention contain preferably 0.01 to 5 wt % of hydantoin and/or at least one hydantoin derivative. It is particularly preferred according to the present invention to use hydantoin derivatives, 5-ureidohydantoin being particularly preferred. Regardless of whether hydantoin or (a) hydantoin derivative(s) is/are used, utilization quantities from 0.02 to 2.5 wt are very particularly preferred, from 0.05 to 1.5 wt % more preferred, 0.075 to 1 wt % and in particular 0.1 to 0.25 wt % (based in each case on the total agent) highly preferred.

The use according to the present invention of polyhydroxy compounds as an active substance with the other components according to the present invention can be particularly preferred. “Polyhydroxy compounds” are to be understood as organic compounds having at least two hydroxy groups.

To be understood thereamong in particular for purposes of the present invention are:
polyols having at least two hydroxy groups, for example trimethylolpropane,
carbohydrates, sugar alcohols, and sugars, as well as salts thereof,
in particular, monosaccharides, disaccharides, trisaccharides, and oligosaccharides, where these can also be present in the form of aldoses, ketoses, and/or lactoses,
amino deoxy sugars, deoxy sugars, thio sugars, where these too can be present in the form of aldoses, ketoses, and/or lactoses.

Very particularly preferred thereamong are monosaccharides having 3 to 8 carbon atoms, such as e.g. trioses, tetroses, pentoses, hexoses, heptoses, and octoses, where these too can be present in the form of aldoses, ketoses, and/or lactoses.

Also preferred are oligosaccharides having up to 50 monomer units. Examples of the polyols according to the present invention that may be mentioned are sorbitol, inositol, mannitol, tetritol, pentitol, hexitol, threitol, erythritol, adonitol, arabitol, xylitol, dulcitol, erythrose, threose, arabinose, ribose, xylose, lyxose, glucose, galactose, mannose, allose, altrose, gulose, idose, talose, fructose, sorbose, psicose, tegatose, deoxyribose, glucosamine, galactosamine, rhamnose, digitoxose, thioglucose, sucrose, lactose, trehalose, maltose, cellobiose, melibiose, gestiobiose, rutinose, raffinose, and cellotriose.

Preferred polyhydroxy compounds are sorbitol, inositol, mannitol, threitol, erythritol, erythrose, threose, arabinose, ribose, xylose, glucose, galactose, mannose, allose, fructose, sorbose, deoxyribose, glucosamine, galactosamine, sucrose, lactose, trehalose, maltose, and cellobiose. Particularly preferably, glucose, galactose, mannose, fructose, deoxyribose, glucosamine, sucrose, lactose, maltose, and cellobiose are used. The use of glucose, galactose, mannose, fructose, sucrose, lactose, maltose, or cellobiose is, however, very particularly preferred.

Among the polyhydroxy compounds having 3 OH groups, glycerol is of outstanding importance.

Regardless of the type of polyhydroxy compound having at least 2 OH groups that is used, agents according to the present invention that contain, based on the weight of the agent, 0.01 to 5 wt %, by preference 0.05 to 4 wt %, particularly preferably 0.05 to 3.5 wt %, and in particular 0.1 to 2.5 wt % polyhydroxy compound(s) are preferred.

Very particularly preferred polyols of the present invention are polyols having 2 to 12 carbon atoms in the molecular skeleton. These polyols can be straight-chain, branched, cyclic, and/or unsaturated. The hydroxy groups are very particularly preferably terminally adjacent or terminally separated from one another by the remainder of the chain Examples of these polyols that may be recited are: glycol, polyethylene glycol up to a molecular weight of 1000 dalton, neopentyl glycol, partial glycerol ethers having a molecular weight of up to 1000 dalton, 1,2-propanediol, 1,3-propanediol, glycerol, 1,2-butanediol, 1,3-butanediol, 1,4-butanediol, 1,2,3-butanetriol, 1,2,4-butanetriol, pentanediols, for example 1,2-pentanediol, 1,5-pentanediol, hexanediols, 1,2-hexanediol, 1,6-hexanediol, 1,2,6-hexanetriol, 1,4-cyclohexanediol, 1,2-cyclohexanediol, heptanediols, 1,2-heptanediol, 1,7-heptanediol, octanediols, 1,2-octanediol, 1,8-octanediol, 2-ethyl-1,3-hexanediol, octadienols, decadienols, dodecanediols, 1,2-dodecanediol, 1,12-dodecanediol, 1,12-dodecanediol having 10 mol EO, dodecadienols.

The polyhydroxy compounds according to the present invention are contained in the compositions in concentrations from 0.01 wt % to 20 wt %, by preference from 0.05 wt % to 15 wt %, and very particularly preferably in quantities from 0.1 wt % to 10 wt %.

In a further embodiment, the agents according to the present invention should additionally contain at least one UV light protection filter. “UV light protection filters” are to be understood as organic substances that are capable of absorbing ultraviolet rays and re-emitting the received energy in the form of longer-wave radiation, e.g. heat. UVB filters can be oil-soluble or water-soluble.

The cosmetic agents can additionally contain further active substances, adjuvants, and additives such as, for example:
structuring agents such as maleic acid and lactic acid,
perfume oils, dimethylisosorbide, and cyclodextrins,
dyes for coloring the agent,
anti-dandruff active substances such as piroctone olamide, zinc omadine, and climbazol,
cholesterol,
complexing agents such as EDTA, NTA, β-alaninediacetic acid, and phosphonic acids,
opacifiers such as latex, styrene/PVP and styrene/acrylamide copolymers,
luster agents such as ethylene glycol mono- and distearate as well as PEG-3 distearate,
pigments,
stabilizing agents for hydrogen peroxide and other oxidizing agents,
propellants such as propane/butane mixtures, N₂O, dimethyl ether, CO₂, and air,
antioxidants.

With regard to further optional components as well as the quantities of those components used, reference is made expressly to the relevant manuals known to one skilled in the art.

As already mentioned, the excellent care-providing effect of the agents according to the present invention is significant in particular because it provides outstanding results even in the presence of oxidizing agents, for example in the context of oxidative hair coloring.

A second subject of the invention is therefore a method for treating hair, in which a cosmetic agent in accordance with claim 1 is applied onto the hair and remains there until the hair is next washed.

It is possible according to the present invention, although not preferred, also to rinse the composition according to the present invention out of the hair again after a contact time. The contact time is preferably from a few seconds to 100 minutes, particularly preferably 1 to 50 minutes, and very particularly preferably 1 to 30 minutes.

The Examples below are intended to explain the subject matter of the present invention without, however, limiting it.

A third subject of the invention is the fact that the cosmetic composition is present as a non-aerosol spray treatment, and is applied onto the hair and remains there until the hair is next washed.

### Examples

All quantity indications are parts by weight unless otherwise noted.

Conditioner

| | C1 | C2 | C3 | C4 | C5 | V1 | V2 |
|---|---|---|---|---|---|---|---|
| Cyclomethicone and dimethiconol | 70.0 | 70.0 | 35.0 | 70.0 | 70.0 | 70.0 | 70.0 |
| Octamethyltrisiloxane | 27.5 | — | — | 27.5 | — | 27.5 | — |
| Cyclopentasiloxane | — | 27.5 | 37.0 | 0 | 27.5 | 0 | 27.5 |
| Natural oil | 0.5 | 0.5 | 0.5 | 0.5 | 0.5 | 0.5 | 0.5 |
| Perfume | 0.5 | 0.5 | 0.5 | 0.5 | 0.5 | 0.5 | 0.5 |
| Dye | 1.0 | 1.0 | 1.0 | 1.0 | 1.0 | 1.0 | 1.0 |
| Acrylic acid ester 1 | 0.5 | 0.5 | 0.5 | — | — | — | — |
| Acrylic acid ester 2 | — | — | — | 0.5 | — | — | — |
| Acrylic acid ester 3 | — | — | — | — | 0.5 | — | — |
| Ethanol | — | — | to 100 | — | — | to 100 | to 100 |

The acrylic acid esters that are used all conform to formula (b1), in which residues R1 to R4 have the following meanings:

| | R1 | R2 | R3 | R4 |
|---|---|---|---|---|
| Acrylic acid ester | phenyl | phenyl | cyano | 2-ethylhexyl |
| 1 | | | | |
| Acrylic acid ester | hydrogen | methoxyphenyl | hydrogen | 2-ethylhexyl |
| 2 | | | | |
| Acrylic acid ester | hydrogen | methoxyphenyl | hydrogen | isoamyl |
| 3 | | | | |

The formulations recited above were stored with five different perfume oils at 45° C. in a light cabinet. Formulations C1 to C5 were each stable for at least 4 weeks without clouding, discoloration, or change in scent. Formulations C1 to C3 were in fact stable for 6 weeks at 45° C. without any change in color or scent. After only one week, compositions V1 and V2 exhibited appreciable and considerable discoloration, were no longer clear, and the scent changed markedly.

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention, it being understood that various changes may be made in the function and arrangement of elements described in an exemplary embodiment without departing from the scope of the invention as set forth in the appended claims and their legal equivalents.

What is claimed is:

1. A cosmetic composition for treating keratinic fibers, comprising:
a. at least one ester oil in a total quantity from 0.01 to 20.0 wt %;
b. at least one silicone in a total quantity from 0.01 to 99 wt %;
c. at least one acrylic acid derivative of formula c) in a total quantity from 0.01 to 5.0 wt % 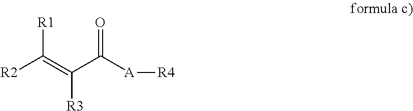
in which residues R1, R2, R3, mutually independently in each case, denote a residue selected from hydrogen, linear and/or branched, saturated and/or unsaturated C1 to C30 alkyl or alkenyl residues, in particular methyl, ethyl, propyl, isopropyl, butyl, isobutyl, tert-butyl, pentyl, amyl, isoamyl, neopentyl, hexyl, isohexyl, 2-ethylhexyl, heptanyl, isoheptanyl, octanyl, isooctanyl, nonyl, decyl, lauryl, myristyl, aryl, phenyl, methoxyphenyl, hydroxyphenyl, methoxybenzyl, hydroxybenzyl, benzyl, naphthyl, or —CN,
A denotes O or —NRS, and
R4 and optionally R5 mutually independently denote a residue selected from hydrogen, linear and/or branched, saturated and/or unsaturated C1 to C30 alkyl or alkenyl residues, in particular methyl, ethyl, propyl, isopropyl, butyl, isobutyl, tert-butyl, pentyl, amyl, isoamyl, neopentyl, hexyl, isohexyl, 2-ethylhexyl, heptanyl, isoheptanyl, octanyl, isooctanyl, nonyl, decyl, lauryl, myristyl, aryl, phenyl, methoxyphenyl, hydroxyphenyl, methoxybenzyl, hydroxybenzyl, benzyl, or naphthyl; and
d. water in a total quantity from 0 to at most 5.0 wt %.

2. The cosmetic composition according to claim 1, wherein the silicone is one or more compounds selected from the group consisting of dimethicones, dimethiconols, and cyclomethicones.

3. The cosmetic composition according to claim 1, wherein A in formula c) denotes an oxygen atom.

4. The cosmetic composition according to claim 1, wherein the ester oil is selected from the group consisting of vegetable oils and natural oils.

5. The cosmetic composition according to claim 1, wherein the ester oil is selected from the group consisting of amaranth seed oil, apricot kernel oil, argan oil, avocado oil, babassu oil, cottonseed oil, pomegranate seed oil, grapefruit seed oil, hazelnut oil, elderberry seed oil, blackcurrant seed oil, jojoba oil, cocoa butter, macadamia nut oil, corn oil, almond oil, marula oil, evening primrose oil, olive oil, orange oil, sea buckthorn pulp oil, sea buckthorn seed oil, sesame oil, shea butter, soy oil, sunflower oil, grapeseed oil, walnut oil, wheat germ oil, wild rose oil, and mixtures thereof.

6. The cosmetic composition according to claim 1, further comprising at least one substance from the group consisting of vitamins, provitamins, and vitamin precursors as well as derivatives thereof.

7. The cosmetic composition according to claim 1, further comprising at least one substance from the group consisting of cationic surfactants and cationic polymers.

8. The cosmetic composition according to claim 1, wherein the water content is at most 0.3 wt %.

9. A method for treating hair, comprising:
applying a composition in accordance with claim 1 onto the hair; and
allowing the composition to remain in the hair until the hair is next washed.
